SEQUENCE LISTING (80 sequences)

SEQ ID NO: 1 - RNA, 30 nt, synthetic construct
  source 1..30: mol_type = other RNA
ttaatacgacctctctgttggattttgaca

SEQ ID NO: 2 - RNA, 22 nt, synthetic construct
  source 1..22: mol_type = other RNA
ggttcgcaacttcacacagagt

SEQ ID NO: 3 - RNA, 22 nt, synthetic construct
  source 1..22: mol_type = other RNA
caggcgtttgttggttgattaa

SEQ ID NO: 4 - RNA, 30 nt, synthetic construct
  source 1..30: mol_type = other RNA
gttttacgacaacgatgttggtttaggaca

SEQ ID NO: 5 - RNA, 22 nt, synthetic construct
  source 1..22: mol_type = other RNA
ggttcggttgttatatacgata

SEQ ID NO: 6 - RNA, 22 nt, synthetic construct
  source 1..22: mol_type = other RNA
ggttcagagagtctcctattta

SEQ ID NO: 7 - RNA, 21 nt, synthetic construct
  source 1..21: mol_type = other RNA
gtggtattcttgctagttaca

SEQ ID NO: 8 - RNA, 21 nt, synthetic construct
  source 1..21: mol_type = other RNA
ggatttgtcttctacaatttg

SEQ ID NO: 9 - RNA, 21 nt, synthetic construct
  source 1..21: mol_type = other RNA
cgaacgctttcttattacaaa

SEQ ID NO: 10 - RNA, 21 nt, synthetic construct
  source 1..21: mol_type = other RNA
cactgttgctacatcacgaac

SEQ ID NO: 11 - RNA, 21 nt, synthetic construct
  source 1..21: mol_type = other RNA
cctagtaataggtttcctatt

SEQ ID NO: 12 - RNA, 21 nt, synthetic construct
  source 1..21: mol_type = other RNA
gcgtgtagcaggtgactcagg

SEQ ID NO: 13 - RNA, 21 nt, synthetic construct
  source 1..21: mol_type = other RNA
gaccgcttctagaaagtgaac

SEQ ID NO: 14 - RNA, 21 nt, synthetic construct
  source 1..21: mol_type = other RNA
caaggacctgcctaaagaaat

SEQ ID NO: 15 - RNA, 21 nt, synthetic construct
  source 1..21: mol_type = other RNA
atggatttgtcttctacaatt

SEQ ID NO: 16 - RNA, 21 nt, synthetic construct
  source 1..21: mol_type = other RNA
cgtagtcgcaacagttcaaga

SEQ ID NO: 17 - RNA, 21 nt, synthetic construct
  source 1..21: mol_type = other RNA
ggtggaccctcagattcaact

SEQ ID NO: 18 - RNA, 21 nt, synthetic construct
  source 1..21: mol_type = other RNA
ggttcaccgctctcactcaac

SEQ ID NO: 19 - RNA, 21 nt, synthetic construct
  source 1..21: mol_type = other RNA
cgggaacgtggttgacctaca

SEQ ID NO: 20 - RNA, 21 nt, synthetic construct
  source 1..21: mol_type = other RNA
gcttatgtgtcaacctatact

SEQ ID NO: 21 - RNA, 21 nt, synthetic construct
  source 1..21: mol_type = other RNA
ggttgaagcagttaattaaag

SEQ ID NO: 22 - RNA, 21 nt, synthetic construct
  source 1..21: mol_type = other RNA
cgatattacgcacaactaatg

SEQ ID NO: 23 - RNA, 21 nt, synthetic construct
  source 1..21: mol_type = other RNA
agaccatgttgacatactagg

SEQ ID NO: 24 - RNA, 21 nt, synthetic construct
  source 1..21: mol_type = other RNA
ggttctttaatctactcaacc

SEQ ID NO: 25 - RNA, 21 nt, synthetic construct
  source 1..21: mol_type = other RNA
ggtggtgttgttcagttgact

SEQ ID NO: 26 - RNA, 21 nt, synthetic construct
  source 1..21: mol_type = other RNA
gctcgtgttgtacgatcaatt

SEQ ID NO: 27 - RNA, 21 nt, synthetic construct
  source 1..21: mol_type = other RNA
agtgtataatgctagtttatt

SEQ ID NO: 28 - RNA, 21 nt, synthetic construct
  source 1..21: mol_type = other RNA
ggtacatgtcagcattaaatc

SEQ ID NO: 29 - RNA, 21 nt, synthetic construct
  source 1..21: mol_type = other RNA
gttagatgatgatagtcaaca

SEQ ID NO: 30 - RNA, 21 nt, synthetic construct
  source 1..21: mol_type = other RNA
gattgtttaggaagtctaatc

SEQ ID NO: 31 - RNA, 21 nt, synthetic construct
  source 1..21: mol_type = other RNA
gtctctagtcagtgtgttaat

SEQ ID NO: 32 - RNA, 21 nt, synthetic construct
  source 1..21: mol_type = other RNA
ctactaatgttgttattaaag

SEQ ID NO: 33 - RNA, 21 nt, synthetic construct
  source 1..21: mol_type = other RNA
ggtgttcttactgagtctaac

SEQ ID NO: 34 - RNA, 21 nt, synthetic construct
  source 1..21: mol_type = other RNA
gaaacaaagtgtacgttgaaa

SEQ ID NO: 35 - RNA, 21 nt, synthetic construct
  source 1..21: mol_type = other RNA
gttagatttcctaatattaca

SEQ ID NO: 36 - RNA, 21 nt, synthetic construct
  source 1..21: mol_type = other RNA
ctgtgatgttgtaataggaat

SEQ ID NO: 37 - RNA, 21 nt, synthetic construct
  source 1..21: mol_type = other RNA
ggagtcaaattacattacaca

SEQ ID NO: 38 - RNA, 21 nt, synthetic construct
  source 1..21: mol_type = other RNA
gcttactctaataactctatt

SEQ ID NO: 39 - RNA, 21 nt, synthetic construct
  source 1..21: mol_type = other RNA
ggtaactgtgatgttgtaata

SEQ ID NO: 40 - RNA, 21 nt, synthetic construct
  source 1..21: mol_type = other RNA
tggtaactgtgatgttgtaat

SEQ ID NO: 41 - RNA, 21 nt, synthetic construct
  source 1..21: mol_type = other RNA
ggtacgttaatagttaatagc

SEQ ID NO: 42 - RNA, 21 nt, synthetic construct
  source 1..21: mol_type = other RNA
gtggtattcttgctagttaca

SEQ ID NO: 43 - RNA, 21 nt, synthetic construct
  source 1..21: mol_type = other RNA
gttaatagttaatagcgtact

SEQ ID NO: 44 - RNA, 21 nt, synthetic construct
  source 1..21: mol_type = other RNA
ggtggaattgctatcgcaatg

SEQ ID NO: 45 - RNA, 21 nt, synthetic construct
  source 1..21: mol_type = other RNA
ggatttgtcttctacaatttg

SEQ ID NO: 46 - RNA, 21 nt, synthetic construct
  source 1..21: mol_type = other RNA
cgaacgctttcttattacaaa

SEQ ID NO: 47 - RNA, 21 nt, synthetic construct
  source 1..21: mol_type = other RNA
cactgttgctacatcacgaac

SEQ ID NO: 48 - RNA, 21 nt, synthetic construct
  source 1..21: mol_type = other RNA
cctagtaataggtttcctatt

SEQ ID NO: 49 - RNA, 21 nt, synthetic construct
  source 1..21: mol_type = other RNA
cgtagtcgcaacagttcaaga

SEQ ID NO: 50 - RNA, 21 nt, synthetic construct
  source 1..21: mol_type = other RNA
ggctactaccgaagagctacc

SEQ ID NO: 51 - RNA, 21 nt, synthetic construct
  source 1..21: mol_type = other RNA
caagatggtatttctactacc

SEQ ID NO: 52 - RNA, 21 nt, synthetic construct
  source 1..21: mol_type = other RNA
gcttatgtgtcaacctatact

SEQ ID NO: 53 - RNA, 21 nt, synthetic construct
  source 1..21: mol_type = other RNA
ggttgaagcagttaattaaag

SEQ ID NO: 54 - RNA, 21 nt, synthetic construct
  source 1..21: mol_type = other RNA
cgatattacgcacaactaatg

SEQ ID NO: 55 - RNA, 21 nt, synthetic construct
  source 1..21: mol_type = other RNA
gattgtttaggaagtctaatc

SEQ ID NO: 56 - RNA, 21 nt, synthetic construct
  source 1..21: mol_type = other RNA
gtctctagtcagtgtgttaat

SEQ ID NO: 57 - RNA, 21 nt, synthetic construct
  source 1..21: mol_type = other RNA
ctactaatgttgttattaaag

SEQ ID NO: 58 - RNA, 21 nt, synthetic construct
  source 1..21: mol_type = other RNA
ggtgttcttactgagtctaac

SEQ ID NO: 59 - RNA, 765 nt, synthetic construct
  source 1..765: mol_type = other RNA
atgaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagtaa

SEQ ID NO: 60 - RNA, 21 nt, synthetic construct
  source 1..21: mol_type = other RNA
ggaagagacaggtacgttaat

SEQ ID NO: 61 - RNA, 21 nt, synthetic construct
  source 1..21: mol_type = other RNA
gaagagacaggtacgttaata

SEQ ID NO: 62 - RNA, 21 nt, synthetic construct
  source 1..21: mol_type = other RNA
ggaagagataggtacgttaat

SEQ ID NO: 63 - RNA, 21 nt, synthetic construct
  source 1..21: mol_type = other RNA
gaagagataggtacgttaata

SEQ ID NO: 64 - RNA, 21 nt, synthetic construct
  source 1..21: mol_type = other RNA
gctgctgtttacagaataaat

SEQ ID NO: 65 - RNA, 21 nt, synthetic construct
  source 1..21: mol_type = other RNA
cggtactattaccgttgaaga

SEQ ID NO: 66 - RNA, 21 nt, synthetic construct
  source 1..21: mol_type = other RNA
ggttccaacggtactattacc

SEQ ID NO: 67 - RNA, 21 nt, synthetic construct
  source 1..21: mol_type = other RNA
cgaagagctaccagacgaatt

SEQ ID NO: 68 - RNA, 21 nt, synthetic construct
  source 1..21: mol_type = other RNA
gagacagaagaaacagcaaac

SEQ ID NO: 69 - RNA, 21 nt, synthetic construct
  source 1..21: mol_type = other RNA
ggatgatttctccaaacaatt

SEQ ID NO: 70 - RNA, 21 nt, synthetic construct
  source 1..21: mol_type = other RNA
ggtggaccctcagattcaact

SEQ ID NO: 71 - RNA, 21 nt, synthetic construct
  source 1..21: mol_type = other RNA
ggacgaagatgacaatttaat

SEQ ID NO: 72 - RNA, 21 nt, synthetic construct
  source 1..21: mol_type = other RNA
ggtgttgtttgtacagaaatt

SEQ ID NO: 73 - RNA, 21 nt, synthetic construct
  source 1..21: mol_type = other RNA
cctcagtgttgacactaaatt

SEQ ID NO: 74 - RNA, 21 nt, synthetic construct
  source 1..21: mol_type = other RNA
ggatgttaactgcacagaagt

SEQ ID NO: 75 - RNA, 21 nt, synthetic construct
  source 1..21: mol_type = other RNA
gcaactgtgttgctgattatt

SEQ ID NO: 76 - RNA, 21 nt, synthetic construct
  source 1..21: mol_type = other RNA
gggtgttaactgcacagaagt

SEQ ID NO: 77 - RNA, 21 nt, synthetic construct
  source 1..21: mol_type = other RNA
ggtatagattgtttaggaagt

SEQ ID NO: 78 - RNA, 68 nt, synthetic construct
  source 1..68: mol_type = other RNA
  stem_loop 31..39
ttaatacgacctctctgttggattttgacattcaagagatgtcaaatccaacagagaggtcgtattaa

SEQ ID NO: 79 - RNA, 53 nt, synthetic construct
  source 1..53: mol_type = other RNA
  stem_loop 23..31
ggttcgcaacttcacacagagtttcaagagaactctgtgtgaagttgcgaacc

SEQ ID NO: 80 - RNA, 53 nt, synthetic construct
  source 1..53: mol_type = other RNA
  stem_loop 23..31
ggttcggttgttatatacgatattcaagagatatcgtatataacaaccgaacc

(19) United States
(12) Patent Application Publication
(10) Pub. No.: US 2024/0368601 A1
(43) Pub. Date: Nov. 7, 2024

(54) NCOVSIRNA DRUG FOR TARGETED DELIVERY SHRNA by RBD, SYNTHESIS METHOD, AND APPLICATION THEREOF

(71) Applicant: Binghuan WENG, Hangzhou (CN)

(72) Inventors: Binghuan WENG, Hangzhou (CN); Hefeng HUANG, Hangzhou (CN); Lin HE, Shanghai (CN); Zhi CHEN, Hangzhou (CN); Hangping YAO, Hangzhou (CN); Wei XU, Hangzhou (CN); Min CHEN, Hangzhou (CN); Yu MA, Hangzhou (CN); Haokun YANG, Hangzhou (CN); Deshuai YU, Hangzhou (CN); Jun YING, Hangzhou (CN)

(21) Appl. No.: 18/661,524

(22) Filed: May 10, 2024

Related U.S. Application Data
(63) Continuation of application No. PCT/CN2022/131458, filed on Nov. 11, 2022.

(30) Foreign Application Priority Data
Nov. 11, 2021 (CN) 202111329883.7
Feb. 8, 2022 (CN) 202210116884.1
May 1, 2022 (CN) 202210491811.0
Jun. 9, 2022 (CN) 202210652488.0
Aug. 1, 2022 (CN) 202210920201.8

(51) Int. Cl. C12N 15/113; A61K 9/127; A61K 39/215; A61K 39/39; A61P 37/04

(52) U.S. Cl. CPC C12N 15/1131; A61K 9/127; A61K 39/215; A61K 39/39; A61P 37/04; C12N 15/111; C12N 2310/14; C12N 2310/3513; C12N 2310/531

(57) ABSTRACT

The present disclosure relates to a synthesis method of A targeted drug nCoVshRNA, using RBD derived from the receptor binding domain of Covid-19 as a targeted delivery carrier, synthesizing shRNA with siRNA selected from common RNAi sequence of various stains; and connecting the plus and antisense strands of the shRNA to the N terminus of the RBD, to obtain a compound with targeted gene drug and macromolecular vaccine; and enabling RBD and shRNA to produce new effects. Among them, shRNA is both a broad spectrum antiviral drug and an immune adjuvant to enhance RBD vaccine; RBD acts as a targeted delivery carrier to avoid the side effects of non-targeted therapy; and RBD is a protein vaccine, and anti-RBD can neutralize the viruses and prevent viruses infection through ACE2.

### CROSS REFERENCE TO RELATED DISCLOSURES

This application is a continuation of International Patent Application No. PCT/CN2022/131458 with a filing date of Nov. 11, 2022, designating the United States, now pending, and further claims priority to Chinese Patent disclosure No. 202111329883.7 filed on Nov. 11, 2021 in the China National Intellectual Property Administration, Chinese Patent disclosure No. 202210116884.1 filed on Feb. 8, 2022 in the China National Intellectual Property Administration, Chinese Patent disclosure No. 202210491811.0 filed on May 1, 2022 in the China National Intellectual Property Administration, Chinese Patent disclosure No. 202210652488.0 filed on Jun. 9, 2022 in the China National Intellectual Property Administration, Chinese Patent disclosure No. 202210920201.8 filed on Aug. 1, 2022 in the China National Intellectual Property Administration, the disclosures of all of which are hereby incorporated by reference.

### SEQUENCE LISTING

The present application contains a sequence listing which was filed electrically in XML format and is hereby incorporated by reference in its entirety. The XML-format sequence listing file was created on May 9, 2024, named as “Sequence_List”, and 73 kb in size.

### TECHNICAL FIELD

The present disclosure relates to the technical field of biopharmaceutical, in particular to a synthesis method of chemical drug nCoVshRNA⋅2RBD.

### BACKGROUND

The structure of coronavirus includes single stranded plus ribonucleic acid acid (ssRNA), spike protein(S), membrane protein (M), envelope protein (E), and nuclear shell protein (N), where S1 sub-unit of the spike protein spike consists of N-terminal domain (S1-NTD) and receptor binding domain (S1-RBD).

Human angiotensin converting enzyme II (ACE2) is a type I transmembrane glycoprotein expressed by target cells susceptible to coronavirus, consisting of 805 amino acids, including the transmembrane region, intracellular carboxyl terminal, and extracellular amino terminal. coronavirus bind to ACE2 of target cells via their S1-RBD, through cell membrane fusion and endocytosis allowing viruses enter target cells expressing ACE2 or containing ACE2 channels.

Based on the relationship that RBD and ACE2 are ligand and receptor, the RBD polypeptide is designed as a targeted delivery carrier, and RBD polypeptide delivers the drug to the target cells expressing ACE2 through RBD, to allow the drug exerts a specific effect on the viruse-infected cells.

RNA interference (RNAi), as an efficient sequence-specific gene silencing technology, is bringing unimaginable disclosure prospects to the treatment of diseases. A variety of siRNA drugs have been examined and approved by the Food and Drug Administration FDA, and siRNA drugs are currently available in the market. However, the existing siRNA drugs often adopt only one viruse strain to design siRNA, apply single-stranded siRNA (antisense RNA) to prepare siRNA drugs, or use a non-targeted delivery carrier for non-specific delivery of siRNA drugs. For the constantly mutated coronavirus, the above siRNA designed based on one viruse strain will go off the target and lose efficency due to the mutation of the coronavirus. Therefore, the siRNA shared by all kinds of strains should be selected from the mutated strains, for the preparation of broad spectrum siRNA drugs targeted on the mutated strains and the to be mutated strains. If a non-targeted delivery carrier is used, siRNA drugs will be delivered to cells that are skipping in expressing ACE2 and are not susceptible to coronavirus, causing side effects of non-targeted delivery. Therefore, targeted delivery carriers that are capable of specifically delivering siRNA to target cells should be designed. More importantly, according to the RNAi mechanism reported by HreA et al., the homologous mRNA silencing efficiendy of the double-stranded RNA that prepared by double-stranded RNA (shRNA/dsRNA) containing both plus and antisense siRNA is 100 times higher than that prepared by single-stranded RNA (antisense RNA), indicating that correct and effective RNAi technology should adopt double-stranded RNA (shRNA/dsRNA) containing both plus and antisense siRNA. Although traditional siRNA drugs based on RNAi mechanism, they are not based on double-stranded RNA. Therefore, the present disclosure synthesized shRNA for RNAi treatment, so that it is theoretically consistent with the RNAi mechanism and the RNAi effect is increased by more than 100 times according to the report of Hre A.

Small interfering RNA, known as siRNA, delivering the siRNA to the cytoplasm regulates gene expression in RNAi ways, specifically degrading the complementary target messenger RNA (mRNA). Because siRNA itself is difficult to cross the cell membrane and is easily degraded by RNA enzymes, lipid nanoparticles encapsulation is traditionally used for non-specific transportation and protection of siRNA. This disclosure syntheses the shRNA based on siRNA, and attaches the RBD polypeptide at the terminals of the shRNA duplex for specific transportation and protection of siRNA.

The prior arts mainly designed the vaccine based on the S1-RBD of Corona Virus Disease 2019 (Covid-19). As reported by the prior arts, the immunogenicity of of inactivated vaccines and subunit vaccines is weak, and immunoadjuvants should be used simultaneously. While siRNA/dsRNA/shRNA may cause an immune response mediated by type I interferon (IFN-α/β) or pro-inflammatory cytokines (IL 6 and TNFα), Among them, IFN-α/β has functions of broading spectrum antiviral effects, enhancing hematopoietic cell proliferation and immune regulation, IL 6 has the functions of inducing B cell differentiation, producing immunoglobulin, promoting the proliferation of T cells and hematopoietic stem cells. Essentially The siRNA/dsRNA/shRNA was an oligonucleotide immunoadjuvant involved in immune activation. The present disclosure syntheses shRNA based on siRNA, and connects RBD polypeptide at the terminals of shRNA, so that the obtained nCoVshRNA⋅2RBD contains RBD-dimer. In terms of RBD vaccine, the RBD-dimer has more compound molecular structure and larger molecular weight than the original single strand RBD. Especially, the immune adjuvant effect of the shRNA makes the vaccine more effective.

In order to solve the above problems, the present disclosure synthesizes shRNA based on siRNA, and connects RBD polypeptide at the terminals of shRNA duplex, to obtain the chemical drug nCoVshRNA⋅2RBD and to target delivery shRNA by RBD, in which shRNA is both an immune adjuvant and a broad spectrum siRNA drug, and RBD is both a targeted delivery carrier and a RBD-dimer vaccine, and can compete with viruses for ACE2 receptors, so as to inhibit viruses infection.

### SUMMARY

The object of the present disclosure is to provide a nCOVsiRNA drug for targeted delivery of shRNA by RBD, a synthesis method and an application thereof. The nCOVsiRNA drug includes shRNA, RBD and liposome, where the shRNA has the dual actions of targeted gene therapy and immune adjuvant, RBD has the roles of targeted delivery, protein vaccine and neutralizing ACE2 receptors, and liposome has the roles of stabilizing shRNA, cell transfection and immune adjuvant.

The object of the present disclosure is implemented through the following technical solutions.

Selecting the target siRNA of anti-variant strains to synthesize shRNA, and binding RBD polypeptide at terminals of shRNA duplex to synthesize nCoVshRNA⋅2RBD, a chemical drug with broad spectrum targeted drug against Covid-19 and a new vaccine carrying immune adjuvant.

Selection the anti variant strain targets: selecting siRNA from common genes of various pathogenic coronavirus and related variant strains, to obtain selected siRNA, the common genes includes conserved genes, ultraconserved genes and/or conserved microsatellite, and the common target siRNA are selected from the various coronavirus and relatived variant strains which are not changed with the viruses variation, to get broad spectrum of anti variant strains effect.

Syntheses of target siRNA of anti-variant strains: synthesizing target siRNA with two complementary oligonucleotides based on the selected siRNA, where the nucleotide number of each complementary oligonucleotide is 21 nt to 25 nt, and synthesizing base sequences to act as spacers.

Syntheses of shRNA: further synthesizing small hairpin shRNA duplexes by the two complementary oligonucleotides siRNA and the base sequences, where, a loop ring of the small hairpin shRNA duplex is formed by the base sequences.

Selection of preferred siRNA: constructing interference carriers based on the small hairpin shRNA duplex, detecting mRNA expression, protein expression and interference effect, selecting preferred siRNA with high silencing efficiency after siRNA design, synthesis, selecting, iterative design and verification.

Syntheses of prefered siRNA and prefered shRNA: synthesizing prefered siRNA and prefered shRNA using preferred siRNA sequence, and performing chemical modifications to increase stability and avoid off-target.

Syntheses of RBD polypeptide or proteins or expressing through RBD genes: synthesizing 319th to 401th amino acid sequence that is located at but not limited to S protein of the coronavirus, synthesizing conserved amino acid sequence that is located at but not limited to sites of N439, V483 and Q493, and synthesizing codon-optimized amino acid sequence.

Syntheses of chemical drug nCoVshRNA⋅2RBD: connecting the prefered shRNA and the RBD by one connecting way that selected from disulfide bond, phosphodiester bond, dithiophosphorate lipid bond, sulfide bond, oxime bond, amide bond or maleimide-thiol bond; or synthesizing RBD-shRNA-RBD compond directly based on a nucleotide sequence of the shRNA and an amino acid sequence of the RBD.

Purification of the RBD-shRNA-RBD: purifing the obtained RBD-shRNA-RBD by high-performance liquid chromatography HPLC, reverse HPLC, or ion exchange chromatography.

Liposome modify of the RBD-shRNA-RBD: preparing liposome modified compound by an attractive force between negatively charged shRNA and positively charged liposome; preparing the liposome modified compound that is internalized by polyethylene glycol PEG, based on a maleimide-sulfydryl bond formed between a sulfydryl of a persulfidation of RBD amino-group and a maleimide of the liposome; preparing the liposome modified compound by carbamate bonds with the amino terminus of RBD and liposome; preparing the liposome modified compound by connecting RBD or RBD fragments to liposome modified siRNA.

Verification of compounds: testing the antiviral effect of the liposome modified compound against two or more different variant strains in vitro cellular level; and observing whether they have broad spectrum anti variant strains effects on targets of conserved genes; and testing whether the liposome modified compound have RNAi effects on targeted delivery of shRNA in animal bodies; and testing the immunological effect of the vaccine; and testing the immune enhancement effects.

The beneficial effects of the present disclosure are listed below:

For the first time, identifing the novel targeted delivery carrier RBD derived from coronavirus receptor binding domain and the target shRNA with broad spectrum anti variant strains derived from coronavirus conserved genes, and synthesizing the RBD and the shRNA as the chemical drug nCoVshRNA⋅2RBD for targeted delivery of shRNA by RBD. Among them, shRNA has effects of broad spectrum anti-variant strains and immune adjuvant, and has effects of of targeted delivery of shRNA, shRNA protection, cell penetrating peptide and protein vaccine. With the mutual promotion of the RBD and the shRNA, further generating many new effects.

One of distinguishing features between the present disclosure and the traditional siRNA drugs is that the present disclosure selects the common target siRNA of various strains from the various coronavirus and relatived variant strains that do not change with the viruses variation, so that the siRNA has a broad spectrum anti-mutation strain effect.

One of distinguishing feature between the present disclosure and the traditional siRNA drug is that the traditional siRNA drugs use plus or antisense siRNA, that is, preparing siRNA drugs by single-stranded siRNA; and the present disclosure synthesizes shRNA by plus and antisense siRNA. According to the RNAi mechanism reported by Hre A, the RNAi effect is increased by more than 100 times. So the disclosure is designed more correctly and more consistent with the RNAi mechanism.

One of distinguishing feature between the present disclosure and traditional siRNA drugs is that the present disclosure delivers shRNA by RBD as a targeted delivery carrier. coronavirus specifically infects target cells expressing ACE2, and there is no existing targeted delivery carrier that specifically delivers siRNA to virus-infected cells but not to uninfected cells. According to the special relationship between the RBD and the ACE2, by connecting the RBD to the shRNA, to allow the RBD generate a new function of targeted delivery of shRNA, thus avoiding the side effects of non-specific delivery.

As siRNA/shRNA is negatively charged, lipid-soluble, not easy to cross the cell membrane, and easily degraded by nuclease, it is difficult to be delivered to the target cytoplasm to produce RNAi. However, after the shRNA and RBD polypeptide are synthesized as compounds, the RBD has properties of cell penetrating peptide, so it can protect the shRNA from degradation by nuclease and make it easier to be delivered to the cytosol through the target cell membrane.

One of the distinguishing feature of the disclosure and the traditional RBD vaccine is that the disclosure synthetizes a RBD-dimer vaccine with 1 part shRNA and 2 parts of RBD, shRNA is used as an immune adjuvant. For nCoVshRNA⋅2RBD synthesized by 2 parts RBD and 1 part shRNA, the total molecular weight of the RBD and the shRNA is more than doubled of the molecular weight traditional single-strand RBD vaccine, and the molecular structure is more compound, and the immunogenicity is stronger. As the main components of immune adjuvants are oligonucleotides and lipids, siRNA or shRNA that are essentially oligonucleotides have immune adjuvant effects to enhance the immune effect of RBD vaccines.

In addition to delay the release speed of shRNA in vivo, prolong efficacy and endocytosis into the slurry, liposome can also serve as an immune adjuvant to enhance the immune effect of RBD protein.

RBD in chemical drug of can complete with the RBD of coronavirus to bind the ACE2 receptors of target cells, thus inhibiting viral infection. In addition, nCoVshRNA⋅2RBD synthesized by 2 parts RBD and 1 part shRNA, the total molecular weight of the RBD and the shRNA is increased, and the molecular structure is more compound, and the immunogenicity is stronger, thus enhancing immunogenicity. The shRNA bound RBD is not easily degraded by enzymes, easily cross the cell membrane, and easily delivered to the target cytoplasm.

In vitro cell experiments shows that the synthesized compound of nCoVshRNA⋅2RBD is effective against two different variant strains, indicating the effects of broad spectrum anti variant strain effects on targets of conserved genes, and in vivo animal experiments shows that the synthesized compound of nCoVshRNA⋅2RBD have the effect of targeted delivery of RNAi, the immune effect of the vaccine and the effect of immune enhancement.

The method of preparing nCoVsiRNA⋅2RBD drug based on binding targeted delivery carrier RBD to the terminals of shRNA duplex of the present disclosure is expected to be applied to the preparation of siRNA gene therapy drugs such as viruses, bacteria, tumors, and genetic diseases.

The nCoVsiRNA⋅2RBD drug of the present disclosure integrates a broad spectrum anti-variant strain targeted drug and a RBD-dimer vaccine. The compound of nCoVsiRNA⋅2RBD adopts binding the coronavirus receptor domain S1-RBD as the carrier, and realizes targeteddelivery a common RNAi target shRNA of the coronavirus variant strains to the ACE2 expressing cell.

The broad spectrum anti-variant strains targeted drugs, refers to the siRNA with a broad spectrum anti-variant strains effect and is designed based on the common RNAi sequence the coronavirus and related variant strains that does not change with the viral variation. The prefered siRNA was then synthesized to the shRNA; connecting the shRNA with the S1-RBD polypeptide which is both the cell penetrating peptide and ACE2 ligand, to optimize the cellular membrane permeability and anti-nuclease stability of the shRNA, further to make shRNA specifically delivered to the ACE2 expressed target cytoplasm more easier, and to silence the target genes that are the ACE2 expressed cells and are susceptible to virus expression.

The S1-RBD-dimer vaccine refers to a new technology line vaccine that connecting 1 part shRNA with 2 part S1-RBD polypeptide, and shRNA has immune adjuvant effect, and the S1-RBD polypeptide have effects of broading spectrum anti-mutation strain and protein antigen, and S1-RBD-dimer vaccine has larger molecular weight, more compound structure and autoimmune adjuvant components.

### BRIEF DESCRIPTION OF THE DRAWINGS

FIG. 1 is a technical flow chat of preparation of nCoVsiRNA drug according to the present disclosure.

FIG. 2 is a schematic diagram of synthesis and application of the present disclosure.

FIG. 3 is a schematic diagram of a compound synthesized of the present disclosure.

FIG. 4 is a schematic diagram of a liposome modified siRNA of the present disclosure.

FIG. 5 is a schematic diagram of a liposome modified shRNA of the present disclosure.

FIG. 6 is a schematic diagram of targeted delivery of shRNA by RBD and the RBD completes with viruses for ACE2 receptors.

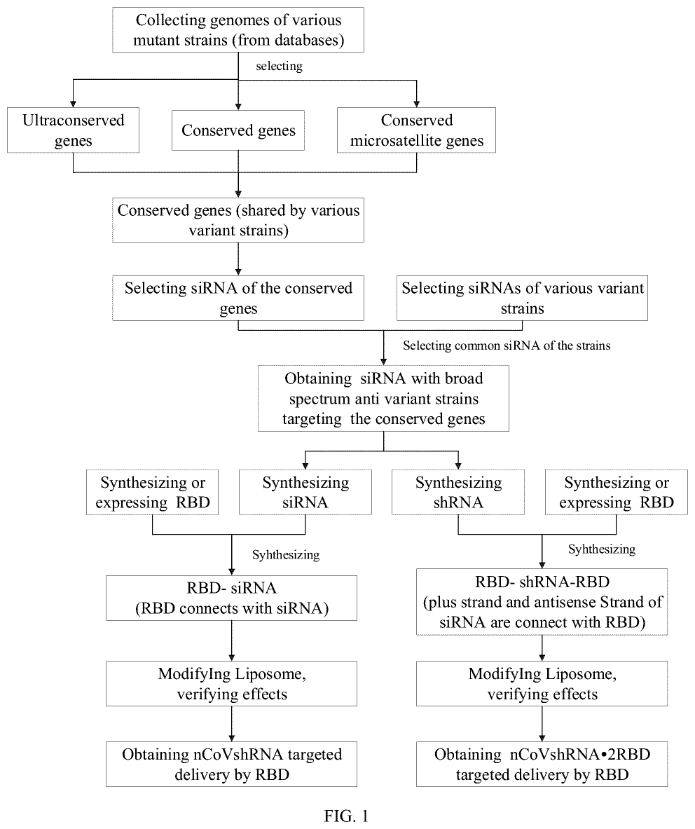

In FIG. 1, after the steps of selecting the conserved genes, selecting target siRNA of broad spectrum anti variant strain based on the conserved genes, synthesizing shRNA, synthesizing RBD, and finally synthesizing nCoVsiRNA drug targeted delivery by RBD.

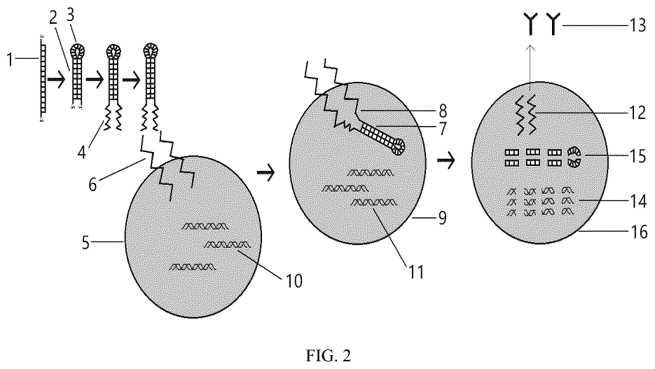

In FIG. 2, 1 is an siRNA that daopts coronavirus conserved genes as target; 2 is plus and antisense strands of the small hairpin shRNA, the shRNA is formed by annealing of 2 complementary oligonucleotides of the siRNA; and 3 is a loop ring is formed by the base sequences and is configured to separate plus and antisense strands; and 4 are two RBD polypeptide, therough N amino group of the RBD polypeptide to connect with the plus and antisense strands of the shRNA; and 5 are the target cells of ACE2 receptors 6 expressing the ACE2, viruses 10 enter the target cells 5 by binding to C terminus of its RBD to N terminus of outer cell membrane of the ACE2 receptors 6. The RBD of the RBD is the same as the RBD of the viruses 10, and binding C terminus of the RBD polypeptide 4 to N terminus that outside of the membrane of the ACE2 receptors 6, to realize targeted delivery of shRNA2 to target cells 5 expressing the ACE2 receptors 6. the target cells 5 are susceptible to infection with viruses 10 because they contain the ACE2 receptors 6. Further, as shown in the target cells 9 where the nCoVsiRNA⋅2RBD drug is entered. 7 and 8 indicate the shRNA and RBD polypeptide, respectively, for the targeted delivery of RBD polypeptide 4 and into the target cells 9 through the ACE2 receptors 6. Further, as shown in the target cells 16 where the nCoVsiRNA⋅2drug is released, as the shRNA7 in the target cell 9 is degraded to the fragments of shRNA 15 in the target cell 16, viruses 10 and long strand RNA 11 are also degraded to small fragment RNA sequences 14 and inactivated. While the RBD polypeptide 8, previously connected to shRNA 7, were also dissociated to fragments of RBD polypeptide 12, thus stimulating the host to produce antibodies 13 of against-RBD polypeptide 12.

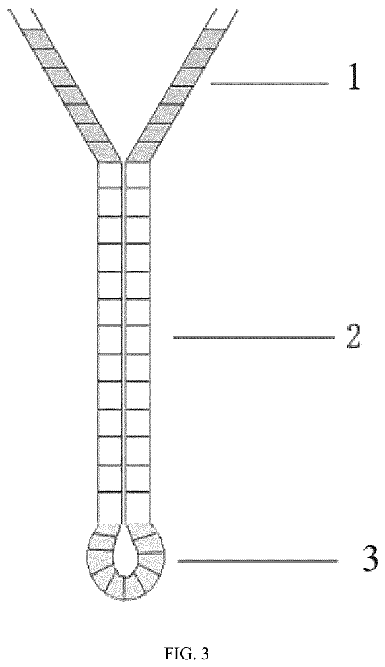

In FIG. 3, 1 is a loop ring, 2 is plus and antisense strands of the small hairpin shRNA, and 4 is two RBD polypeptide (protein), and the two RBD polypeptide 4 are connected to the plus and antisense strand of the shRNA 2. The shRNA is protected by the RBD polypeptide 4 and is delivered specifically by the RBD to the ACE2 receptors 6, and then specifically enters the target cytoplasm as the RBD cross the cell membrane of the ACE2 receptors to degrade viral target genes.

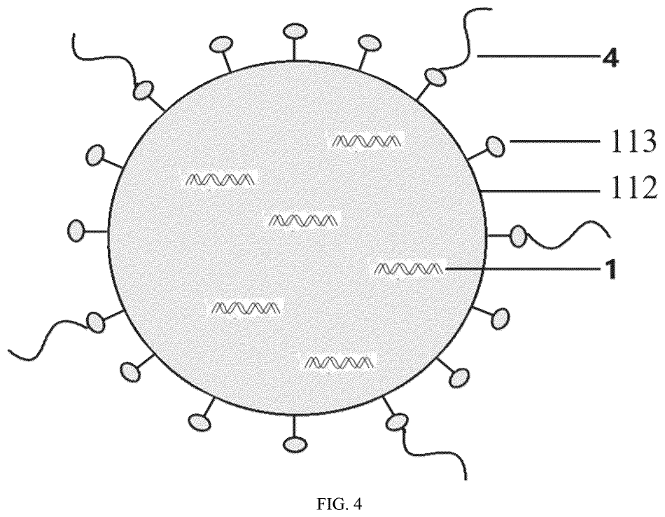

In FIG. 4, where the nCoVsiRNA⋅2RBD drug has not entered the target cell 5, 1 is the siRNA coated by liposome, and 112 is liposome layer, and 113 is PEG layer, and 4 is the RBD polypeptide. Where siRNA acts as RNAi, liposome protect siRNA and cause cell endocytosis, PEG delay the siRNA release and prolong the effective time, and RBD polypeptide plays the dual role of targeted delivery of siRNA and vaccine.

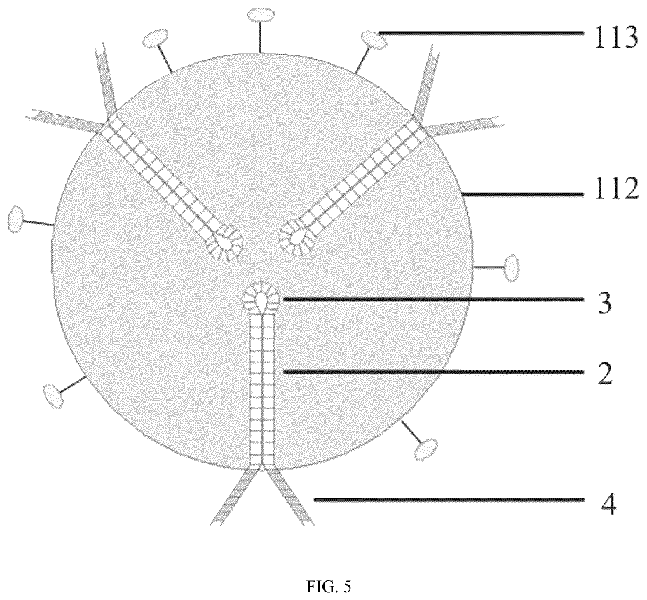

In FIG. 5, where the nCoVsiRNA⋅2RBD drug is entered the target cell 9, 3 is the loop ring, 2 is plus and antisense strands of the small hairpin shRNA, 4 is two RBD polypeptide (protein), and the two RBD polypeptide are respectively connected to the plus and antisenseh RNA. 112 is the liposome layer, 113 is the PEG layer, and the shRNA2 is coated by the liposome layer 112. Among them, shRNA acts as RNAi, liposome protect shRNA and cause cell endocytosis, PEG delay the siRNA release and prolong the effective time, and RBD polypeptide plays the dual role of targeted delivery of siRNA and vaccine.

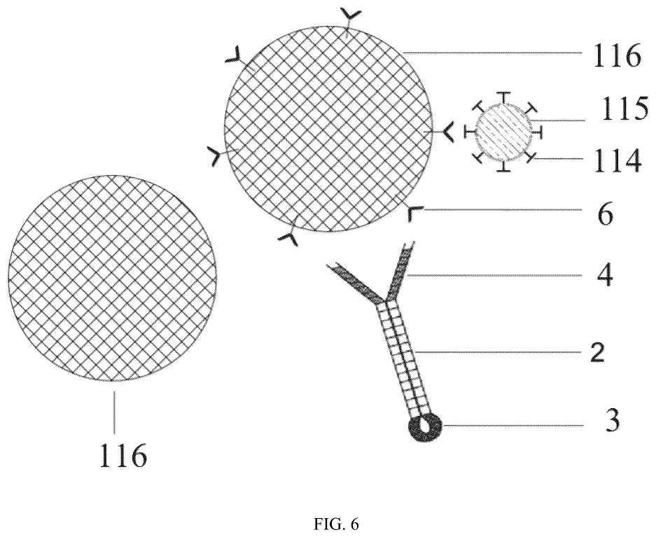

In FIG. 6, 3 is the loop ring, 2 are plus and antisense strands of the small hairpin shRNA, and 4 is the RBD polypeptide, and 6 is ACE2 receptors, 114 is the RBD of Covid-19, 115 is Covid-19, and 16 is the target cells expressing the ACE2 receptors, and 116 is cell that do not express the ACE2 receptors. The targeted delivery carrier RBD polypeptide 4 specifically delivered the RNAi drug shRNA to target cells 16 expressing ACE2 receptors 6, and Covid-19 5 also infected target cells by binding of RBD polypeptide 4 to ACE2 receptors 6. Since the targeted delivery of RBD polypeptide 4 allows shRNA 2 specifically into target cells 16 but not susceptible to viruses 116, the RBD competes with the viral RBD for ACE2 receptors 6, thus competing to inhibit viral infection.

### DETAILED DESCRIPTION OF THE EMBODIMENTS

Please refer to FIGS. 1-6, examples of specific methods of the disclosure are described in detail, but these exemplary descriptions do not constitute any limitation on the scope of protection defined by the claims of this disclosure.

### I. Design of siRNA Targeting Ultraconserved Genes, Conserved Genes, or Conserved Microsatellite

1. Design of the siRNA Targeting the Ultraconserved Genes, the Conserved Genes, and the Conserved Microsatellite.

As shown in the technical flow chat of FIG. 1, downloading a whole genome (cDNA) sequence of B coronavirus genus (especially Covid-19 and its variant strains) from the Genbank database (http://www.NCBI.nlm.nih.gov/genome/); searching for the longest public sub sequence in the whole genome sequence, to obtain ultraconserved genes or conserved genes; and comparing the sequence alignment of the whole genome downloaded from the Genbank database using a Clustal W software, to detect similarities between the different sequences; and selecting sequences of conserved microsatellite; and using a molecular evolutionary genetic analysis software MEGA 6.0, constructing an amino acid germ line molecular evolution tree for the downloaded coronavirus amino acid sequence by a Neighbour-Jioning N-J method; and analyzing and optimizing the variation characterizations of molecules in amino acid sequence; and inferring sequences of the conserved gene, and taking the sequences of the conserved gene as a basis for siRNA synthesis.

Obtaining the following 3 sequences which are longest and the second longest sub conserved subsequences, which are 22 bp to 30 bp, and are equivalent to small RNA, but higher organisms do not contain these three sequences, especially humans. The specific sequences are as follows:

SEQ ID NO. 1 (Subsequence 1) =
ttaatacgacctctctgttggattttgaca (30 bp);
SEQ ID NO. 2 (Subsequence 2) =
ggttcgcaacttcacaca gagt (22 bp);
SEQ ID NO. 3 (Subsequence 3) =
caggcgtttgttggttgattaa (22 bp).

Obtaining the following 3 sequences which are longest and the second longest conserved subsequences, which are 22 bp to 30 bp, and are equivalent to small RNA, but higher organisms do not contain these three sequences, especially humans. The specific sequences are as follows:

SEQ ID NO. 4 (Subsequence 1) =
gttttacgacaacgatgttggtttaggaca (30 bp);
SEQ ID NO. 5 (Subsequence 2) =
ggttcggttgttatatacgata (22 bp);
SEQ ID NO. 6 (Subsequence 3) =
ggttcagagagtctcctattta (22 bp).

Obtaining the following five conserved microsatellite sites which are repeated multiple times by nucleotides, and the microsatellite sites are CTCTCT, AGAGAG, AAAAAAA, TATATA and CACACA.

2. Design of the Common Target siRNA of the Covid-19 Variant Strains.

Downloading whole genome (cDNA) sequences of β coronavirus genus (especially Covid-19 and its variant strains) from the Genbank database (http://www.NCBI.nlm.nih.gov/genome/); Obtaining multiple candidate siRNA sequences with a length of about 19nt by using Ambion's shRNA online design software (http://www.ambion.com/techlib/misc/siRNAtools.html) or other software such as DSIR; and selecting prefered siRNA according to a Tm value of RNA binding and the specific alignment results. Therefore, the RNAi sequences (siRNA) of each strain and the common RNAi sequences, that is, target siRNA are obtained from the E, M, N, ORF1ab and S genes of the 18 Covid-19 strains; and the common siRNA of each strain is shown in Table 1, and its sequence is marked as SEQ ID NO.7-40. For example, siRNA labeled in Tables 2 to 5 (SEQ ID NO.41 to 58) is the common target siRNA of NC_045512.2, Delta strain and Omicron strain, and the siRNA without sequence number marker is respective RNA sequence (siRNA). Thus, it can be seen that the earliest variant of NC_045512.2 strain is Delta strain and the nearest Omicron sequence, but in each strain, except for their unique target interference sequence siRNA (unmarked part), it exists the common conserved sequence SEQ ID NO.41 to 58 remains unchanged and theoretically has targeted interference effect.

TABLE 1
Common target siRNA selected from 18 Covid-19 variants (SEQ ID
NO.7~40)

| | | | Common target siRNA | | | |
|---|---|---|---|---|---|---|
| Variant | | | of the variant strains | | Off- | |
| strains | region | position | (ss sequence) | score | targets | SEQ ID NO. |
| (1) | E gene | 7 | gtggtattcttgctagttaca | 95.1 | 0 | SEQ ID NO. 7 |
| B.1.617.1, | M gene | 35 | ggatttgtcttctacaatttg | 95.7 | 0 | SEQ ID NO. 8 |
| (2) | | 146 | cgaacgctttcttattacaaa | 94.8 | 0 | SEQ ID NO. 9 |
| B.1.1.529 | | 130 | cactgttgctacatcacgaac | 94.8 | 0 | SEQ ID NO. 10 |
| (3) B.1.1.7, | | 6 | cctagtaataggtttcctatt | 94.7 | 0 | SEQ ID NO. 11 |
| (4) P.1, | | 181 | gcgtgtagcaggtgactcagg | 94.3 | 0 | SEQ ID NO. 12 |
| (5) BA.2, | | 18 | gaccgcttctagaaagtgaac | 93.3 | 0 | SEQ ID NO. 13 |
| (6) B.1.351, | | 109 | caaggacctgcctaaagaaat | 91 | 0 | SEQ ID NO. 14 |
| (7) B.1.525, | | 33 | atggatttgtcttctacaatt | 91.0 | 0 | SEQ ID NO. 15 |
| (8) | N gene | 137 | cgtagtcgcaacagttcaaga | 100.7 | 0 | SEQ ID NO. 16 |
| B.1.617.2, | | 12 | ggtggaccctcagattcaact | 99.9 | 0 | SEQ ID NO. 17 |
| (9) C.1.2, | | 56 | ggttcaccgctctcactcaac | 91.4 | 0 | SEQ ID NO. 18 |
| (10) | | 39 | cgggaacgtggttgacctaca | 90.4 | 0 | SEQ ID NO. 19 |
| B.1.6211, | ORF1ab | 7687 | gcttatgtgtcaacctatact | 106.6 | 0 | SEQ ID NO. 20 |
| (11)(11) | gene | 8304 | ggttgaagcagttaattaaag | 105.1 | 0 | SEQ ID NO. 21 |
| B.1621, | | 8613 | cgatattacgcacaactaatg | 104.6 | 0 | SEQ ID NO. 22 |
| (12) P.2, | | 10519 | agaccatgttgacatactagg | 104.4 | 0 | SEQ ID NO. 23 |
| (13) N.9, | | 6719 | ggttctttaatctactcaacc | 104.4 | 0 | SEQ ID NO. 24 |
| (14) C.37.1, | | 1805 | ggtggtgttgttcagttgact | 104.3 | 0 | SEQ ID NO. 25 |
| (15) C.37, | | 1619 | gctcgtgttgtacgatcaatt | 104.2 | 0 | SEQ ID NO. 26 |
| (16) | | 11328 | agtgtataatgctagtttatt | 103.9 | 0 | SEQ ID NO. 27 |
| B.1.427, | | 4932 | ggtacatgtcagcattaaatc | 103.8 | 0 | SEQ ID NO. 28 |
| (17) | | 2935 | gttagatgatgatagtcaaca | 103.5 | 0 | SEQ ID NO. 29 |
| B.1.3512, | S gene | 4 | gattgtttaggaagtctaatc | 103.6 | 0 | SEQ ID NO. 30 |
| (18) | | 29 | gtctctagtcagtgtgttaat | 101.5 | 0 | SEQ ID NO. 31 |
| B.1.3513 | | 33 | ctactaatgttgttattaaag | 99.8 | 0 | SEQ ID NO. 32 |
| | | 6 | ggtgttcttactgagtctaac | 99.2 | 0 | SEQ ID NO. 33 |
| | | 68 | gaaacaaagtgtacgttgaaa | 97.1 | 0 | SEQ ID NO. 34 |
| | | 155 | gttagatttcctaatattaca | 96.7 | 0 | SEQ ID NO. 35 |
| | | 7 | ctgtgatgttgtaataggaat | 94.9 | 0 | SEQ ID NO. 36 |
| | | 271 | ggagtcaaattacattacaca | 94.6 | 0 | SEQ ID NO. 37 |
| | | 12 | gcttactctaataactctatt | 94.6 | 0 | SEQ ID NO. 38 |
| | | 2 | ggtaactgtgatgttgtaata | 94.1 | 0 | SEQ ID NO. 39 |
| | | 1 | tggtaactgtgatgttgtaat | 94.1 | 0 | SEQ ID NO. 40 |

TABLE 2
The candidate siRNA sequences of the Covid-19 NC_045512.2, DELTA,
and OMICRON strain E genes.

| | | | | | Off- | |
|---|---|---|---|---|---|---|
| siRNA ID (E) | | position | ss sequence (sirna) | score | targets | SEQ ID NO. |
| NC_045512.2 | 1 | 16 | ggaagagacaggtacgttaat | 101.7 | 0 | SEQ ID NO. 60 |
| | 2 | 17 | gaagagacaggtacgttaata | 98 | 0 | SEQ ID NO. 61 |
| | 3 | 26 | ggtacgttaatagttaatagc | 96.4 | 0 | SEQ ID NO. 41 |
| | 5 | 68 | gtggtattcttgctagttaca | 95.1 | 0 | SEQ ID NO. 42 |
| | 6 | 31 | gttaatagttaatagcgtact | 95 | 0 | SEQ ID NO. 43 |
| DELTA | 1 | 16 | ggaagagacaggtacgttaat | 101.7 | 0 | SEQ ID NO. 60 |
| | 2 | 17 | gaagagacaggtacgttaata | 98 | 0 | SEQ ID NO. 61 |
| | 3 | 26 | ggtacgttaatagttaatagc | 96.4 | 0 | SEQ ID NO. 41 |
| | 5 | 68 | gtggtattcttgctagttaca | 95.1 | 0 | SEQ ID NO. 42 |
| | 6 | 31 | gttaatagttaatagcgtact | 95 | 0 | SEQ ID NO. 43 |
| OMICRON | 1 | 16 | ggaagagataggtacgttaat | 102.3 | 0 | SEQ ID NO. 62 |
| | 2 | 17 | gaagagataggtacgttaata | 98.6 | 0 | SEQ ID NO. 63 |
| | 3 | 68 | gtggtattcttgctagttaca | 95.1 | 0 | SEQ ID NO. 42 |
| | 4 | 31 | gttaatagttaatagcgtact | 95 | 0 | SEQ ID NO. 43 |
| | 5 | 26 | ggtacgttaatagttaatagc | 94.9 | 1 | SEQ ID NO. 41 |

TABLE 3
The candidate siRNA sequences of M genes of Covid-19 NC_045512.2,
DELTA and OMICRON.

| | | | | | Off- | |
|---|---|---|---|---|---|---|
| siRNA ID (M) | | position | ss sequence (sirna) | score | targets | SEQ ID NO. |
| NC_045512.2 | 1 | 200 | gctgctgtttacagaataaat | 103.4 | 0 | SEQ ID NO. 64 |
| | 2 | 13 | cggtactattaccgttgaaga | 97.4 | 0 | SEQ ID NO. 65 |
| | 3 | 230 | ggtggaattgctatcgcaatg | 97.2 | 0 | SEQ ID NO. 44 |
| | 4 | 90 | ggatttgtcttctacaatttg | 95.7 | 0 | SEQ ID NO. 45 |
| | 5 | 518 | cgaacgctttcttattacaaa | 94.8 | 0 | SEQ ID NO. 46 |
| | 6 | 502 | cactgttgctacatcacgaac | 94.8 | 0 | SEQ ID NO. 47 |
| | 9 | 61 | cctagtaataggtttcctatt | 94.7 | 0 | SEQ ID NO. 48 |
| DELTA | 1 | 200 | gctgctgtttacagaataaat | 103.4 | 9 | SEQ ID NO. 64 |
| | 2 | 13 | cggtactattaccgttgaaga | 97.4 | 1 | SEQ ID NO. 65 |
| | 3 | 230 | ggtggaattgctatcgcaatg | 97.2 | 0 | SEQ ID NO. 44 |
| | 4 | 90 | ggatttgtcttctacaatttg | 95.7 | 0 | SEQ ID NO. 45 |
| | 5 | 518 | cgaacgctttcttattacaaa | 94.8 | 0 | SEQ ID NO. 46 |
| | 6 | 502 | cactgttgctacatcacgaac | 94.8 | 0 | SEQ ID NO. 47 |
| | 9 | 61 | cctagtaataggtttcctatt | 94.7 | 0 | SEQ ID NO. 48 |
| OMICRON | 1 | 200 | gctgctgtttacagaataaat | 103.4 | 9 | SEQ ID NO. 64 |
| | 2 | 5 | ggttccaacggtactattacc | 99.5 | 0 | SEQ ID NO. 66 |
| | 3 | 13 | cggtactattaccgttgaaga | 97.4 | 1 | SEQ ID NO. 65 |
| | 4 | 230 | ggtggaattgctatcgcaatg | 97.2 | 0 | SEQ ID NO. 44 |
| | 5 | 90 | ggatttgtcttctacaatttg | 95.7 | 0 | SEQ ID NO. 45 |
| | 6 | 518 | cgaacgctttcttattacaaa | 94.8 | 0 | SEQ ID NO. 46 |
| | 7 | 502 | cactgttgctacatcacgaac | 94.8 | 0 | SEQ ID NO. 47 |
| | 9 | 61 | cctagtaataggtttcctatt | 94.7 | 0 | SEQ ID NO. 48 |

TABLE 4
The candidate siRNA sequences of N genes of the Covid-19 NC_045512.2,
DELTA, and OMICRON strains.

| | | | | | Off- | |
|---|---|---|---|---|---|---|
| siRNA ID (N) | | position | ss sequence (sirna) | score | targets | SEQ ID NO. |
| NC_045512.2 | 1 | 260 | cgaagagctaccagacgaatt | 101.1 | 1 | SEQ ID NO. 67 |
| | 2 | 1150 | gagacagaagaaacagcaaac | 100.1 | 36 | SEQ ID NO. 68 |
| | 3 | 563 | cgtagtcgcaacagttcaaga | 100.7 | 0 | SEQ ID NO. 49 |
| | 4 | 1198 | ggatgatttctccaaacaatt | 100.2 | 12 | SEQ ID NO. 69 |
| | 5 | 50 | ggtggaccctcagattcaact | 99.9 | 1 | SEQ ID NO. 70 |
| | 6 | 251 | ggctactaccgaagagctacc | 99.9 | 0 | SEQ ID NO. 50 |
| | 14 | 306 | caagatggtatttctactacc | 95.6 | 0 | SEQ ID NO. 51 |
| DELTA | 1 | 260 | cgaagagctaccagacgaatt | 101.1 | 1 | SEQ ID NO. 67 |
| | 2 | 1150 | gagacagaagaaacagcaaac | 101.1 | 36 | SEQ ID NO. 68 |
| | 3 | 563 | cgtagtcgcaacagttcaaga | 100.7 | 0 | SEQ ID NO. 49 |
| | 4 | 1198 | ggatgatttctccaaacaatt | 100.2 | 12 | SEQ ID NO. 69 |
| | 5 | 50 | ggtggaccctcagattcaact | 99.9 | 1 | SEQ ID NO. 70 |
| | 6 | 251 | ggctactaccgaagagctacc | 99.9 | 0 | SEQ ID NO. 50 |
| | 14 | 315 | caagatggtatttctactacc | 95.6 | 0 | SEQ ID NO. 51 |
| OMICRON | 1 | 251 | cgaagagctaccagacgaatt | 101.1 | 1 | SEQ ID NO. 67 |
| | 2 | 1141 | gagacagaagaaacagcaaac | 101.1 | 36 | SEQ ID NO. 68 |
| | 3 | 554 | cgtagtcgcaacagttcaaga | 100.7 | 0 | SEQ ID NO. 49 |
| | 4 | 1189 | ggatgatttctccaaacaatt | 100.2 | 12 | SEQ ID NO. 69 |
| | 5 | 50 | ggtggaccctcagattcaact | 99.9 | 1 | SEQ ID NO. 70 |
| | 6 | 242 | ggctactaccgaagagctacc | 99.9 | 0 | SEQ ID NO. 50 |
| | 14 | 306 | caagatggtatttctactacc | 95.6 | 0 | SEQ ID NO. 51 |

TABLE 5
The candidate siRNA sequences of ORF1ab genes of the Covid-19
NC_045512.2, DELTA, and OMICRON strains.

| | | | | | Off- | |
|---|---|---|---|---|---|---|
| siRNA ID (ORF) | | position | ss sequence (sirna) | score | targets | SEQ ID NO. |
| NC_045512.2 | 1 | 13351 | ggacgaagatgacaatttaat | 111.5 | 0 | SEQ ID NO. 71 |
| | 2 | 5654 | ggtgttgtttgtacagaaatt | 107.7 | 0 | SEQ ID NO. 72 |
| | 3 | 17851 | cctcagtgttgacactaaatt | 107.2 | 0 | SEQ ID NO. 73 |
| | 4 | 7687 | gcttatgtgtcaacctatact | 106.6 | 0 | SEQ ID NO. 52 |
| | 9 | 8304 | ggttgaagcagttaattaaag | 105.1 | 0 | SEQ ID NO. 53 |
| | 13 | 8610 | cgatattacgcacaactaatg | 104.6 | 0 | SEQ ID NO. 54 |
| DELTA | 1 | 13351 | ggacgaagatgacaatttaat | 111.5 | 7 | SEQ ID NO. 71 |
| | 2 | 5654 | ggtgttgtttgtacagaaatt | 107.7 | 3 | SEQ ID NO. 72 |
| | 3 | 17851 | cctcagtgttgacactaaatt | 107.2 | 6 | SEQ ID NO. 73 |
| | 4 | 7687 | gcttatgtgtcaacctatact | 106.6 | 0 | SEQ ID NO. 52 |
| | 9 | 8304 | ggttgaagcagttaattaaag | 105.1 | 0 | SEQ ID NO. 53 |
| | 13 | 8613 | cgatattacgcacaactaatg | 104.6 | 0 | SEQ ID NO. 54 |
| OMICRON | 1 | 13339 | ggacgaagatgacaatttaat | 111.5 | 7 | SEQ ID NO. 71 |
| | 2 | 5654 | ggtgttgtttgtacagaaatt | 107.7 | 3 | SEQ ID NO. 72 |
| | 3 | 17839 | cctcagtgttgacactaaatt | 107.2 | 6 | SEQ ID NO. 73 |
| | 4 | 7684 | gcttatgtgtcaacctatact | 106.6 | 0 | SEQ ID NO. 52 |
| | 9 | 8301 | ggttgaagcagttaattaaag | 105.1 | 0 | SEQ ID NO. 53 |
| | 13 | 8610 | cgatattacgcacaactaatg | 104.6 | 0 | SEQ ID NO. 54 |

TABLE 6
The candidate siRNA sequences of S genes of the Covid-19 NC_045512.2,
DELTA, and OMICRON strains.

| | | | | | Off- | |
|---|---|---|---|---|---|---|
| siRNA ID (S) | | position | ss sequence (sirna) | score | targets | SEQ ID NO. |
| NC_045512.2 | 1 | 1837 | ggatgttaactgcacagaagt | 105.8 | 0 | SEQ ID NO. 74 |
| | 2 | 1359 | gattgtttaggaagtctaatc | 103.6 | 0 | SEQ ID NO. 55 |
| | 3 | 1074 | gcaactgtgttgctgattatt | 101.9 | 0 | SEQ ID NO. 75 |
| | 4 | 29 | gtctctagtcagtgtgttaat | 101.5 | 0 | SEQ ID NO. 56 |
| | 8 | 366 | ctactaatgttgttattaaag | 99.8 | 0 | SEQ ID NO. 57 |
| | 13 | 1646 | ggtgttcttactgagtctaac | 99.2 | 0 | SEQ ID NO. 58 |
| DELTA | 1 | 1359 | gattgtttaggaagtctaatc | 103.6 | 0 | SEQ ID NO. 55 |
| | 2 | 1837 | gggtgttaactgcacagaagt | 102.4 | 0 | SEQ ID NO. 76 |
| | 3 | 1353 | ggtatagattgtttaggaagt | 102 | 0 | SEQ ID NO. 77 |
| | 5 | 29 | gtctctagtcagtgtgttaat | 101.5 | 0 | SEQ ID NO. 56 |
| | 9 | 366 | ctactaatgttgttattaaag | 99.8 | 0 | SEQ ID NO. 57 |
| | 13 | 1646 | ggtgttcttactgagtctaac | 99.2 | 0 | SEQ ID NO. 58 |
| OMICRON | 1 | 1350 | gattgtttaggaagtctaatc | 103.6 | 0 | SEQ ID NO. 55 |
| | 2 | 1828 | gggtgttaactgcacagaagt | 102.4 | 0 | SEQ ID NO. 76 |
| | 3 | 1065 | gcaactgtgttgctgattatt | 101.9 | 1 | SEQ ID NO. 75 |
| | 4 | 29 | gtctctagtcagtgtgttaat | 101.5 | 0 | SEQ ID NO. 56 |
| | 8 | 360 | ctactaatgttgttattaaag | 99.8 | 0 | SEQ ID NO. 57 |
| | 12 | 1637 | ggtgttcttactgagtctaac | 99.2 | 0 | SEQ ID NO. 58 |

3. Selecting siRNA Targeting Ultraconserved Genes, Conserved Genes or Conserved Microsatellite.

Using Clustal W software or other software, performing a sequence alignment between the sequences the designed genes of ultraconserved genes, conserved genes and conserved microsatellite and the sequence of conventional selected; detecting the similarities between different sequences; and designing pairs of siRNA for ultraconserved genes, conserved genes or conserved microsatellite, and RNAi target site (designing siRNA targeting ultraconserved genes, conserved genes or conserved microsatellite target siRNA).

(1) SiRNA targeting the ultraconserved genes and conserved microsatellite (S1/S2):

SEQ ID NO. 1 (Subsequence 1) =
ttaatacgacctctctgttggattttgaca (30 bp);
SEQ ID NO. 2 (Subsequence 2) =
ggttcgcaacttcacacagagt (22 bp).

(2) siRNA targeting conserved genes and conserved microsatellite (S3/S4):

SEQ ID NO. 5 (Subsequence 3) =
ggttcggttgttatatacgata (22 bp);
SEQ ID NO. 6 (Subsequence 4) =
ggttcagagagtctcctattta (22 bp).

Through the above design, to obtain siRNA targeting the ultraconserved genes and conserved microsatellite and theoretically resistant to coronavirus variant strain, and named them ad siRNA1/2/3/4.

### II. Validation the Common Target siRNA

1. Syntheses of the siRNA/shRNA

According to the RNAi mechanism, when the siRNA effectively interferes with the mRNA expression of S gene, it will form a non infectious S protein deficient virus. When siRNA effectively interferes with mRNA expression of N gene, it suppresses viral packaging and replication. When siRNA effectively interferes with mRNA expression of ORF 1a or 1b genes, it can affect the synthesis of viral RNA polymerase (RdRp) or protein processing enzyme (3 CLpro). However, the M and E genes are the membrane genes of the viruses, and the defects in inhibiting the viruses may not be obvious. Therefore, the present disclosure selects siRNA targeting on N gene (SEQ ID NO.16 to 18, SEQ ID NO. 49 to 51), siRNA targeting on ORF 1 ab gene (SEQ ID NO. 20 to 22, SEQ ID NO. 52 to 54) and siRNA targeting the S gene (SEQ ID NO. 30 to 32, SEQ ID NO. 56 to 58), and the SEQ ID NO. 1 to 2, SEQ ID NO.5 to 6 for the synthesis. Designing shRNA templates to express the hairpin structure according to the pSilencer4.1.CMV.neo the polyclonal restriction sites of the interference carrier, each shRNA template consists of two complementary single-stranded DNA, and a length of each complementary oligonucleotide is 55 bp. And a DNA duplex is formed with complementary single-stranded DNA, DNA duplex with sticky ends at BamH I and Hind III cleavage sites is formed after annealing, and the DNA duplex is configured to connect with a linearized pSilencer4.1.CMV.neo. The siRNA was then synthesized by the designed siRNA and its shRNA template.

2. Construction of the shRNA Expression Carrier.

The shRNA synthesized as above was connected with the linearized interference carrier pSilencer4.1.CMV.neo, and performing ligation and characterization; and constructing shRNA expression plasmid, transformed DH5a, to obtain separate shRNA expression carriers.

3. Validation of the effects of the shRNA expression (interference) carriers.

Based on the synthetic siRNA/shRNA and relative expression plasmid, performing synthetic or PCR amplification to corresponding target genes, constructing a fluorescent tag carrier; and performing a co-transfected with II type alveolar epithelial cells (AEC2s) or 293T cells with shRNA expression plasmids for identification. PCR amplification is routinely performed as follows:

Design of primer: designing upper and downstream primers, and a start code was added to a 5′ terminal of the upstream primer to clone the amplified product into pEGFP-N1, and the 5′ terminal of the primer was added to the homologous arm for homologous recombination with the carrier.

Amplification of targeted gene: performing gene amplification, product recovery and purification according to the gene amplification reaction system and reaction conditions provided by Shanghai Biotech kit, to obtain amplification products.

Linearization of pEGFP-N1: resuscitating DH5a species containing pEGFP-NI plasmid, extracting plasmid based on kit, and performing enzyme digestion after measuring the concentration; and identifying the linearized pEGFP-N1 carrier by and 0.8% agarose gel electrophoresis; and recovering the linearized pEGFP-N1 carrier.

connecting the amplified targeted gene to the fluorescent tag carrier (pEGFP-N1): connecting the amplified targeted gene to the fluorescent tag carrier pEGFP-N1 by the homologous recombination kit from Kingsley Corporation, which can be stored at −20° C. for standby or be converted immediately.

Validating interference effect of the shRNA carrier: performing process of 293T cells cotransfection with the interference carrier (pSilencer-shRNA) and fluorescent tag carrier (pEGFP-N/S/ORF1ab), a mass ratio of the interference carrier and fluorescent tag carrier was 1:2; and setting a control group; and observing fusion expression of GFP protein after 48 h; and evaluating the interference effect according to the fluorescence intensity.

Flow cytometry detection: to quantitatively analyze the interference effects of different carriers, the proportion of fluorescent protein expressing cells in the total number of cells was analyzed by flow cytometry.

Westernbolt Analysis: \textcircled{1} cell collection and lysis: cells were lysed with RIPA. \textcircled{2} SDS-PAGE protein electrophoresis: preparing SDS-PAGE glue, adding the sample to 2xSDS buffer in equal volume, boiling in water for 5 min, taking ice bath for 2 min, 12000×g, 10 min. \textcircled{3} Western blot Detection: performing membrane transfer, sealing, primary antibody binding, washing, secondary antibody binding and color development, and observing results.

Detection of mRNA by RT-PCR: measuring a relative expression of target genes in the transfected cells by relative fluorescence quantitative RT-PCR method. According to the standard curve, calculating relative expression level of viral gene mRNA (targeted gene copy number/B-actin copy number) based on B-actin reference gene.

4. Obtaining siRNA/shRNA with High Silencing Efficiency.

After the above steps of design, synthesis, selection, iterative design, resynthesis and validation at the cellular level, the sequences of siRNA with high silencing efficiency are as follows: SEQ ID NO.1 (named shRNA1, the same below), SEQ ID NO.2 (shRNA2), SEQ ID NO.5 (shRNA3); and SEQ ID NO.16 (shRNA4), SEQ ID NO.49 (shRNA5) of the targeting the N gene; the SEQ ID NO.21 (shRNA6), SEQ ID NO.52 (shRNA7) of the targeting the ORF1ab gene; and the SEQ ID NO.30 (shRNA8) targeting the S gene; and the silencing efficiency was 78%, 76%, 88%, 89%, 89%, 84%, 91% and 90%, respectively.

### III. Synthesis of the shRNA

according to the selected common targets (SEQ ID NO. 1 to 58, preferably shRNA1 to 8), each shRNA is synthesized by two complementary oligonucleotides siRNA, where the nucleotide number of each complementary oligonucleotide is 19 nt to 25 nt, and synthesizing base sequences of 9 nt to act as spacers. Further synthesizing small hairpin shRNA duplexes by the two complementary oligonucleotides siRNA and the base sequences, where, a loop ring of the small hairpin shRNA duplex is formed by the base sequences. Each single strand of the shRNA duplex can connect to the PBD polypeptide or protein, respectively. For example, synthesizing 5′-ttaatacgacctctctgttggattttgacattcaagagatgtcaaatccaacagagaggtcgtattaa-3′ (shRNA1, as shown in SEQ ID NO.78), 5′-ggttcgcaacttcacacagagtttcaagagaactctgtgtgaagttgcgaacc-3′ (shRNA2, as shown in SEQ ID NO.79) and 5′-ggttcggttgttatatacgatattcaagagatatcgtatataacaaccgaacc-3′ (shRNA3, as shown in SEQ ID NO.80) by SEQ ID NO.1. SEQ ID NO.2 and SEQ ID NO.5. Where “TTCAAGAGA” is a loop ring, the sequences located in left side and right side are plus strand and antisense strand, respectively, and to connect the RBD protein or RBD polypeptide at a 3′ terminal and/or 5′ terminal. Similarly, selecting other siRNA with high silencing efficiency were preferred, and synthesizing shRNA.

### IV. Design and Synthesis of the Targeted Delivery Carrier RBD

Obtaining the amino acid sequence corresponding to the SEQ ID NO.59 sequence, and the specific sequence of SEQ ID NO.59 is as follow:

atgaatattacaaacttgtgcccttttggtgaagtttttaacgccacca
gatttgcatctgtttatgcttggaacaggaagagaatcagcaactgtgt
tgctgattattctgtcctatataattccgcatcattttccacttttaag
tgttatggagtgtctcctactaaattaaatgatctctgctttactaatg
tctatgcagattcatttgtaattagaggtgatgaagtcagacaaatcgc
tccagggcaaactggaaagattgctgattataattataaattaccagat
gattttacaggctgcgttatagcttggaattctaacaatcttgattcta
aggttggtggtaattataattacctgtatagattgtttaggaagtctaa
tctcaaaccttttgagagagatatttcaactgaaatctatcaggccggt
agcacaccttgtaatggtgttgaaggttttaattgttactttcctttac
aatcatatggtttccaacccactaatggtgttggttaccaaccatacag
agtagtagtactttcttttgaacttctacatgcaccagcaactgtttgt
ggacctaaaaagtctactaatttggttaaaaacaaatgtgtcaatttca
acttcaatggtttaacaggcacaggtgttcttactgagtctaacaaaaa
gtttctgcctttccaacaatttggcagagacattgctgacactactgat
gctgtccgtgatccacagacacttgagtaa

RBD polypeptide have the dual role of targeted delivery carrier and recombinant protein vaccine. Because the coronavirus cause infection by specifically binding to the ACE2 receptors through RBD, the relationship between RBD and ACE2 is ligands and receptors, thus drugs can be targeted by RBD to the viruses-infected cells and arrived the cytoplasm. In addition, prior arts usually design RBD protein vaccines based on viral infection characteristics of the binding of RBD and ACE2, so the synthetic RBD plays double role of a targeted delivery carrier and a protein vaccine. A a schematic diagram of targeted delivery of shRNA by RBD and the RBD completes with viruses for ACE2 receptors shown in FIG. 6. In FIG. 6, 3 is the loop ring, 2 are plus and antisense strands of the small hairpin shRNA, and 4 is the RBD polypeptide, and 6 is ACE2 receptors, 114 is the RBD of Covid-19, 115 is Covid-19, and 16 is the target cells expressing the ACE2 receptors, and 116 is cell that do not express the ACE2 receptors. The targeted delivery carrier RBD polypeptide 4 specifically delivered the RNAi drug shRNA to target cells 16 expressing ACE2 receptors 6, and Covid-19 5 also infected target cells by binding of RBD polypeptide 4 to ACE2 receptors 6. Since the targeted delivery of RBD polypeptide 4 allows shRNA 2 specifically into target cells 16 but not susceptible to viruses 116, the RBD competes with the viral RBD for ACE2 receptors 6, thus competing to inhibit viral infection.

1. Design and synthesis of amino acid sequence of RBD: collecting S protein gene sequences of SARS-CoV, MERS, and SARS-CoV-2 based on Global influenza shared avian database (GISAID) and the Genbank database; performing an amino acid systematic evolution tree or sequence homology analysis, to determine amino acid sequence sites N439, V483 and Q493 that can bind to human ACE2 receptors and are not prone to variation. In addition, with the features of SARS CoV S protein consists of 1255 amino acids, can be enzymatically digested into S1 receptors binding region (RBD) and S2 membrane fusion region, and RBD is located at 319th to 510th (AA319-510) of amino acid sequence of S protein, and RBD binds to the N terminal of outer membrane of ACE2 by C terminal, and RBD can enter targeted cells alone through ACE2, and removal of glycosylation connected on N terminal of RBD S protein does not affect the function of RBDS protein, and three N-glycosylated residues (N331, N343, N360) are existed in RBD of SARS-CoV-2 (aa.331-550); and tryptophan, histidine, ornithine, lysine and arginine, which constitutes the peptide chain, all have multiple N terminals, which helps to design the synthesis of RBD and connect the RBD to liposome or shRNA.

2. Synthesis of RBD: the method of synthesizing polypeptide usually through peptide bonds that formed by dehydration and condensation of two amino acids, and polypeptide is formed by connecting multiple amino acid residues through peptide bonds. The polypeptide is entrusted a third-party company to synthesis. A peptide synthesizer is used to automatically synthesize amino acid sequence at 319th to 510th sites of S protein, conserved amino acid sequence at sites of N439, V483 and Q493, and codon-optimized amino acid sequence. And the basic method is as follows: adding amino acid one by one according to a order of the amino acid sequence of the synthetic peptide, to make the peptide chain residues gradually extended from the C terminal to the N terminal, and each amino acid residue is condensated in a form of one terminal protection and another terminal activation; and removing temporary protection groups on the amino group after each peptide elongation cycle, until all the condensation of amino acid sequence of the targeted peptide is done. At present, the common reaction principle of solid-phase synthesis of polypeptide is to add the required amino acids in the closed explosion-proof glass reactor, and the order of adding the required amino acids is from the C termina carboxyl end to the N terminal-amino of the polypeptide sequence, performing a synthesis reaction, and getting the polypeptide finally. The main steps include: \textcircled{1} deprotection: removing the protective group of the amino group with alkaline solvent; \textcircled{2} activation and cross-linking: activating the next amino acid, to form a peptide bond between the activated monomer carboxyl group and the free amino group; repeating these two steps until the peptide synthesis is done.

### V. Synthesis of Compound by shRNA and RBD

For the compound synthesized from shRNA and RBD, the shRN is Gene therapy drug, RBD acts as protein vaccine and targeted delivery of shRNA, the compound (RBD-shRNA-RBD) has dual identity of a targeted drug and a vaccine.

1. Design of RBD-shRNA-RBD: as shown in FIG. 2 and FIG. 3, based on the synthetic sequences of shRNA and RBD, the one end of the plus and antisense strand of the siRNA targeting the viral conserved gene is connected to the loop ring (5′-TTCAAGAGA-3′), and the other end connects to the glycosylation site of the RBDS protein N, respectively, forms the “RBD-siRNA plus strand-loop ring-siRNA antisense strand-RBD”. Where, two of the complementary plus and antisense strands form duplexes, but the two RBD polypeptides do not form duplexes, so a hairpin linker shRNA with two peptides RBD and loop rings is formed. As the RBD can connect viruses receptors ACE2 through the C terminal and enter the target cytoplasm by ACE2, and the binding of polypeptide and siRNA can increase the permeability, stability and interference effect, so this design has effects of RBD targeted delivery, membrane peptide, protein antigen and competitive receptors, not only can stable and effectively deliver CoVsiRNA to targeted cells that are viruses infection for targeted interference, and itself as a recombinant protein to stimulate immune antibodies, and can compete with the viruses to bind with targeted cell receptors ACE2 to inhibit viruses infection.

2. Synthesis of RBD-shRNA-RBD: As a choice, the synthesis process can be entrusted to a profession company. Adopting a traditional synthesis method of polypeptide and oligonucleotides. Coupling the polypeptide and the oligonucleotides into conjugates in forms of oxime bond, amide bond, sulfur ether bond, disulfide bond, phosphoryl bond, bond, acylurete bond, phosphodiester bond, dithiophosphorotide bond, maleidyl-sulfhydryl bond, etc.; and cross-linking the polypeptide with the plus strand (5′ terminal, 3′ terminal) and the antisense chain (3′ terminal) of the oligonucleotidesthe by strong covalent bonds, loose ionic bonds, hydrophobic bonds, or carboxyl bonds with spaced arms, to synthesize polypeptide-oligonucleotide conjugates (POCs). At present the most commonly used POCs synthesis method is covalent cross-linking-liquid phase fragment synthesis method, and it has been widely used in the synthesis of various POCs, the main steps are as follows: synthesizing polypeptide and oligonucleotides on the solid phase matrix, respectively; and then peeling the polypeptide and the oligonucleotides from the solid phase matrix, simultaneously; coupling the peeled polypeptide and oligonucleotides in solution by reactive active groups. The steps of synthesizing POCs mainly include: \textcircled{1} maleimide-sulfhydryl coupling: modifying maleimide on the polypeptide or oligonucleotides, and modifying the sulfhydryl group on another monomer; adding the modified polypeptide or oligonucleotides to the same solution to obtain POCs; \textcircled{2} Disulfide bond or thioether bond coupling: the thioether bond coupling includes the reaction of halogenate on haloacetamide and the addition of sulfhydryl Michael to maleimide; where disulfide bond coupling can involve direct oxidation by two sulfhydryl groups, or by coupling a sulfhydryl group to an oligomer containing a thiol group, disulfide bond synthesis of siRNA and polypeptide conjugate is commonly used; \textcircled{3} Oxime bond coupling: the aldehyde group reacts with the amino group to produce the oxime, the reaction conditions were mild and high efficiency, and can directly generate the coupling products of double-stranded DNA bound specific polypeptide; and the oxime can also be used to connect two polypeptide to both 5′ terminal and 3′ terminal of the oligonucleotides through bifunctionalized oligonucleotides, polypeptide or carbohydrates; this method does not require various protection methods and can be completed in one step, and is used for synthesis the “peptide-oligonucleotide-peptide” products, to introduce aldehyde groups at both 5′ terminal and 3′ terminal of the oligonucleotides; and then reaction with the hydroxylamine-modified polypeptide, to obtain a “peptide-oligonucleotide-peptide”, and the obtained yield is higher; the one-step reaction of such bifunctionalized oligonucleotides with polypeptide does not require any protective strategy and crosslinkers, in the condition of slightly acid can get a higher yield; \textcircled{4} amide bond coupling: obtaining the product directly using the oligomers containing activated carboxylate or thioester reaction with another modified amino group of the polymer; \textcircled{5} hydrazone bond coupling: introducing the hydraazine group to the polypeptide, adding citrate buffer solution between pH 3 and 5, rereaction with oligonucleotides modified with acetyl aldehyde group, to obtain POCs with hydrazone bond connected.

3. Purification of the RBD-shRNA-RBD: Chromatography has always been one of the most commonly used methods for the purification and analysis of polypeptide and oligonucleotide conjugates. According to a compoundity of the conjugate to be purified, to choose different chromatography methods for separation. The main methods include high performance liquid chromatography (HPLC), reverse high performance liquid chromatography (RP-HPLC), ion exchange chromatography (IEC, mostly anion exchange chromatography), or using two or several of them in series, according to the operation instructions.

According to the selection and synthesis of shRNA and the RBD synthesis, can be obtained including but not limited to siRNA drug synthesized by SEQ ID NO.1 to 58, the compound of RBD-shRNA-RBD, RBD-siRNA or S-siRNA, synthesized by RBD ploypeptide or S protein polypeptide and sequences of SEQ ID NO.1 to 58, and a compound modified by liposome.

The compounds synthesized in this application are RBD-sh RNA (1 to 8)-RBD, RBD-siRNA, and S-siRNA.

### VI. The Liposome Modification of the Compound

As shown in FIGS. 4 to 5, liposome modification includes adsorption between positively charged liposome and negatively charged shRNA, forming thiol-maleimide bond by thiolation of the RBD amino group and the liposome, or forming carbamate bond between the amino terminal group of the RBD and the liposome. By wrapping of liposome DC-chol/DOPE or Lip, to obtain RBD-shRNA (1-8)/Lip-RBD (abbreviated as RBD₂-sh RNA (1-8)/Lip), RBD-siRNA/Lip and S-siRNA/Lip.

The first embodiment: preparing the liposome-modified compound by liposome DOTAP/Chol

### (1) Synthesis of the RBD-shRNA-RBD

In this application, the RBD-shRNA-RBD compound is synthesized by shRNA and RBD.

### (2) Preparation of Lipid Solution

DOTAP (MW=698.5): 10 mg/ml, weighing 100 mg powders of DOTAP [N-1-(2, 3-di-oleoyloxy) propyl)-N,N,N-trimeth ylammoniumethyl sulfate] accurately, adding the powders to a 10 ml volumetric flask and adding chloroform solution to a scale line.

Chol (MW=386): 5 mg/ml, weighing 50 mg of Chol powders [Methoxy-polyethylene glycol-distearoyl phosphatidyi-ethanolamine] accurately, adding the Chol powders to a 10 ml volumetric flask, and then adding chloroform solution to the scale line.

m-PEG2000-DSPE (MW=2787): 10 mg/ml, weighing 10 mg powders of m-PEG2000-DSPE (Maleimide derivatized polyethylene glycol-distearoyl phosphatidyl-ethanolamine), adding 1 ml DEPC water, and then performing a vortex and sonicated for 1 min, to obtain the required solution after complete dissolution.

Mal-PEG2000-DSPE (MW=2941.6): 10 mg/ml, weighing 10 mg powders of Mal-PEG2000-DSPE (Maleimide derivatized polyethylene glycol-distearoyl phosphatidyl-ethanolamine), adding them to 1 ml DEPC water, and then performing a vortex and sonicated for 1 min, to obtain the required solution after complete dissolution.

### (3) Preparation Liposomal DOTAP/Chol (Lipid-Film Method)

Adopting lipid-film method to prepare liposomal DOTAP/Chol, a lipid concentration was 10 mM, and the main steps are as follows: based on the amount ti be used to prepare 1 ml of the liposome DOTAP/Chol, taking Chloroform solutions of DOTAP and Chol, adding them to a 500 ml triangular flask at a ratio of DOTAP: Chol=1:1 (M:M) and adding 3 ml to 4 ml of chloroform solution; performing a vacuum rotary evaporation at 37° C. for 45 min to 60 min, to obtain a homogeneous lipid film; and blowing all traces of chloroform with high purity nitrogen; and adding 1 ml DEPC of water to vibrate and shake, to wash the lipid film from the bottle wall and obtain lipid suspension; After sufficient hydration, ultrasound 1 min, sequentially through 400 nm, 200 nm, 80 nm, and 50 nm polycarbonate membranes, 10 tims to 20 times each, to obtain the liposome DOTAP/Chol.

### (4) Thiolation of the RBD-shRNA-RBD

2-IT (Traut'S reagent) is a commonly used reagent for protein thiolation, and can be performed at the position of glycosylation of the N junction of the RBD S protein, The steps are as follows: taking the RBD-shRNA-RBD and 2-IT (Traut'S reagent, 2-iminothiolane-HCl), mixing evenly with a mole ratio of 2-IT to RBD-shRNA-RBD=200:1; reaction for 2 h at room temperature; removing the excess 2-IT by dialysis; and using sufficient dialysate every time (1×PBS, 5 mM EDTA, PH=7.4); and saving at 4° C. and performing the dialysis overnight with low-speed magnetic stirring, and changing dialysate every 6 h to 8 h, 2 times in all; and determining concentration and degree of thiolation of thiolated antibodies by BCA and determining the protein concentration and degree of thiolation by Ellman, respectively.

### (5) Preparation of Liposome-Modified Compound by the Liposome DOTAP/Chol

(A) Preparation by using Negatively Charged siRNA/shRNA Adsorbing Positively Charged Liposome

\textcircled{1} Taking 120 μl of DOTAP/Chl liposome (10 mM), and adding 20 μl RBD-shRNA-RBD (about 2 μg/μ1,40 μg) with 11 μl of DEPC water; and standing at room temperature for 10 min, to obtain the RBD-shRNA-RBD compound which is modified with liposome.

\textcircled{2} Taking siRNA 90 μl (24 μg, 20 mM), shRNA 90μl (24 μg, 10 mM), and/or RBD-shRNA-RBD 100 μl (about 10 μg/μ 1, 200 μg), adding DEPC water 57.6 μl; and standing at room temperature for 10 min; and adding 600 μl DOTAP/Chl liposome (50 mM) to obtain liposome-coated siRNA, shRNA, and/or RBD-shRNA-RBD compound.

\textcircled{3} mixing the liposome-coated RBD-shRNA-RBD compound prepared by step \textcircled{1} with equal amounts of liposome-coated siRNA, shRNA and/or RBD-shRNA-RBD compound prepared by step \textcircled{2}, to obtain liposome-coated RBD-shRNA/siRNA-RBD compound containing free siRNA/shRNA.

### (B) Preparation was Performed by the Cross-Linking Reaction of the Sulfhydryl Group in the RBD and the Maleimide in the PEG.

To increase the cycle time and targeting specificity of liposome, various liposome prepared from (A) were PEG functionalized and further modified by RBD, to obtain compounds that are modified by PEG and liposome, and the compound adopts RBD as ligand.

Taking 6.36 μl, 9.53 μl, and 12.7 μl of 10 mg/ml of MAL-DSPE-PEG, and adding them to the liposome compound RBD-shRNA/siRNA-RBD prepared by step (A) (mix the two respectively), respectively, 50° C. water bath for 10 min; standing at room temperature for 10 min; adding about 200 μg of the RBD-shRNA-RBD as described above, to induce cross-linking reaction between the thiol group on the sulfamino group of the RBD and the maleimide in MAL-DSPE-PEG, to obtain PEG liposome compound RBD-shRNA/siRNA-RBD of RBD modified with 5 mol % PEG, 7.5 mol % PEG and 10 mol % PEG, respectively; that is, the compound is composed by an electrostatic adsorption of the lipossome DOTAP/Chol to the siRNA, the shRNA and/or the RBD-shRNA-RBD by, and then, coating the MAL-DSPE-PEG at an outer layer; finally, the RBD-shRNA-RBD was connected to the MAL-DSPE-PEG, where RBD acts as targeted delivery carrier, protein antigen and stabilizing siRNA/shRNA; and liposome and PEG protect the siRNA/shRNA, decrease the release speed of siRNA/shRNA/RBD, and intracellular transfection of siRNA/shRNA or vaccine adjuvant.

A second embodiment: preparing a liposome-modified compound by a liposomal liposomal.

When pH>8, the amino terminus of the RBD reacts with pNP-PEG-DPPE (PEG-PE) to form a stable carbamate ester bond compound, and quantitatively inserts into the outer membrane of the liposome to prepare liposome-modified compound. The present embodiment prepares the liposome-modified compound which targeted delivery carrier RBD by siRNA.

### (1) Synthesis of RBD

The RBD or its fragments were synthesized by the RBD synthesis method described above.

(2) Synthesis of pNP-PEG-DPPE

Taking 10 ml of 20 mg/mL DPPE (Dipalmitoyl Phosphoethanolamine) chloroform solution into a 50 mL round bottom flask; adding 0.65 mL of triethylamine (TEA); and taking 4.0g of 200 mg/mL (pNP) 2-PEG3400 (Polyethylene glycol 3400 dip-nitrobenzene carbonate) chloroform solution to add to the above solution; and performing processes of blow nitrogen, seal, avoid light, stirring at room temperature under magnetic overnight, vacuum steam solvent, vacuum remove the residual chloroform; and then adding 100 mL of 0.01 mol/L HCl solution, ultrasonic treatment to form a transparent micelle solution. Where the 0.01 mol/L HCl aqueous solution was eluted and separated by CL-4B Sepharose to remove the unreacted (pNP) 2-PEG3400 and to remove the released pNP; and then collecting eluates containing pNP-PEG-DPPE micelles; and then freeze-dried, and qualitatively and quantified the pNP-PEG-DPPE using TLC, HPLC, MS and NMR.

(3) Synthesis of RBD-PEG-DPPE: taking pNP-PEG-DPPE 100 mg to 10 mL chloroform and making the pNP-PEG-DPPE completely dissolved; placing in a 50 mL flask and removing chloroform to form lipid film; performing a vacuum drying to dissolve the residual chloroform; adding 25 mg RBD in 0.01 mol/L HCl 4 mL to a flask with inner wall lipid film, incubate at room temperature for 30 min, and disperse the lipid film with light shaking. 20 mL of 10 mud/L (pH 9.0) of Tris buffer was mixed, nitrogen protection and incubated overnight at 4° C. The sample was then placed in a dialysis bag with a molecular mass of 5 kD, dialyzed in 10 mmol/L (pH 7.4) Tris buffer for about 4 h, and then dialysis by deionized water at 4° C. for 24 h. Removing the solution in the bag, freeze-dried and stored in a minus 20° C. refrigerator.
(4) Synthesis of RBD-siRNA/liposomal: mixing ePC (egg yolk phospholipid), Ch (cholesterol), PEG2000-DSPE (distearoethanolamine polyethylene glycol 2000) and DOTAP (dioleoyltrimethymepropane) chloroform solution at a molar ratio of 60:34:3.0:3.0. To mark the lipid membrane, adding Rho-PE to the above mixture and to remove chloroform to form a lipid membrane, where a mass mole ratio of the Rho-PE is 0.1%. Taking a certain amount of siRNA dissolved in ultrapure water treated with DEPC, a amount of siRNA should completely neutralize the positive charge carried by DOTAP. The phospholipid membranes were hydrated with an aqueous solution containing siRNA in a 50° C. water bath for 30 min, to form liposome wrapped with siRNA. Using a manual extrusion device (Avanti Polar Lipids), the initially formed liposome were passed polycarbonate core pore membrane (Whatman) for 10 times over 0.4 μm and 0.1 μm to, to obtain liposome with uniform particle size. Taking an appropriate amount of RBD-PEG-DPPE and making the RBD-PEG-DPPE dissolved in methanol, placing in a flask, drying by nitrogen to form a membrane, and adding the prepared liposome suspension a 37° C. water bath for 2 h, so that the RBD-PEG-DPPE was directionally inserted into the outer membrane of the liposome. A molar ratio of RBD to the liposome in the total liposome is generally 0.5% ˜1.0% (which can be adjusted appropriately). The properties of PEG-coated liposome coated with siRNA with RBD modifications were examined by dynamic laser scattering, cryoetching electron microscopy, and nucleic acid electrophoresis.

### VII. Verification of the Compound (nCOVsiRNA)

1. Verifying the Broad-Spectrum Antiviral Effects of Targeting Conserved Genes In Vitro

### (1) Preparation of Virus Solution

Adding the viral strains to the DMEM medium (10% FBS) with Vero E6 cells grown to a confluence of 30%, and continuing to cultivate at a incubator of 36° C., 5% CO₂ for 5 days to 7 days until a cytopathic effect (CPE) appeared, isolating the viruses, and mixed isolated viruses to virus solution of 10³˜10⁵ TCID₅₀/ml with the culture medium. Accordingly, prepare two variant strains of Covid-19 B.1.617.1 and Covid-19 B.1.617.2 to verify whether the compound is simultaneously effective against two or more variant viruses containing the same conserved genes, and to prove whether the shRNA of the present disclosure has a broad-spectrum antiviral effect targeting conserved genes.

(2) Co-Culture of the Compound (nCOVsiRNA) and Viruses

Setting up experimental groups and control groups, respectively, to test the effects of the compound RBD2-shRNA (1-8)/Lip, RBD-siRNA/Lip and S-siRNA/Lip against Covid-19 B.1.617.1 and Covid-19 B.1.617.2. Each group was seeded with 8-well plates and a Vero-E6 cell number of each well is 2×10⁵, and 2 mL DMEM of medium (10% FBS) is added to each well; and co-culturing the Vero-E6 cell and the Covid-19 at 36° C., 5% CO₂ until reach a confluence of 30% (after 24 h); replacing the culture medium, and adding the test compounds, B.1.617.1 and B.1.617.2 strains.

The experimental groups includes: RBD₂-shRNA1 (/lip) group (0.1 nmol RBD₂-shRNA1 (/lip)+0.6 ml virus solution), RBD₂-shRNA2(/lip) group (0.1 nmol RBD₂-shRNA2 (/lip)+0.6 ml virus solution), and so on, RBD₂-shRNA8 (/lip) group (0.1 nmol RBD₂-shRNA8 (/lip)+0.6 ml virus solution), and RBD-siRNA (/lip) group (0.1 nmol RBD-siRNA (/lip)+0.6 ml virus solution). The control groups included: naked sh RNAI group (0.1 nmol naked sh RNA1−0.6 ml virus solution), naked sh RNA2 group (0.1 nmol naked sh RNA2+0.6 ml virus solution), and naked sh RNA3 group (0.1 nmol naked sh RNA3+0.6 ml virus solution), and naked siRNA group (0.1 nmol naked siRNA+0.6 ml viruses solution), and RBD control group (0.1 nmol RBD+0.6 ml viruses solution), positive control group (0.6 ml viruses solution), and negative control group (0.6 ml DMEM culture medium) (Table 1-6).

The culture was continued, and then supernatants were taken from each group after 1 hour, 24 hours and 72 hours of incubation, and diluted in 1:4, 1:121, 1:36, 1:108, 1:324, 1:972, 1:2916, 1:8748 times, and detecting by RT-PCR.

### (3) Real-Time Fluorescence RT-PCR Detection of Viral RNA in Each Group

Viral nucleic acid extraction and nucleic acid (ORF1ab/N) detection operation according to the kit instructions.

### (4) Viruses RNA Test Results

\textcircled{1} B.1.617.1 test results: as shown in Table 7, after the cells of each group were cultured for 1 h, the viral RNA test results of the negative control group were negative, the RNA test titer of the positive control group was 1:36, and the titer of the RNA test results of all other groups was 1:12. As shown in Table 8, after 24 h of culture in each group, the viral RNA test result of the negative control group was still negative, the RNA test titer of the positive control group was 1:2916, and the RNA test titer of the 4 control groups was 1:972˜2:2916, while the RNA test titer of the experimental group was 1:36˜1:108, which was significantly lower than that of the control group (p<0.01). As shown in Table 9, after 72 h of culture, the viral RNA test results of the negative control group were still negative, the RNA test titer of the positive control group was>1:8748, the RNA test titer of the control group was 1:2916 to 1:8748, and the RNA test titer of the experimental group was 1:108 to 1:324, which was significantly lower than that of the control groups (p<0.01).

RBD₂-shRNA (4-8) test results in Tables 7a, 8a and 9a match the RBD₂-shRNA (1-3) test results in Tables 7, 8 and 9. When comparing with the positive control groups. All have significant effects on inhibiting viruses.

Tables 7-9 shows that the experimental groups have obvious anti-B.1.617.1 effects, indicating that shRNA or siRNA connected to RBD could be delivered to target cells for RNA interference, while shRNA or siRNA not connected to RBD could not enter target cells and cannot play the role of RNA interference, in addition, RBD also has certain antiviral effects.

TABLE 7
Test results (+/−) of RNA RT-PCR viruses after co-culturing
the compounds with B.1.617.1 strain for 1 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | − | − | − | − | − | − |
| shRNA1/lip | | | | | | | | |
| RBD₂- | + | + | − | − | − | − | − | − |
| shRNA2/lip | | | | | | | | |
| RBD₂- | + | + | − | − | − | − | − | − |
| shRNA3/lip | | | | | | | | |
| RBD- | + | + | − | − | − | − | − | − |
| siRNA/lip | | | | | | | | |
| naked | + | + | − | − | − | − | − | − |
| shRNA1 | | | | | | | | |
| naked | + | + | − | − | − | − | − | − |
| shRNA2 | | | | | | | | |
| naked | + | + | − | − | − | − | − | − |
| shRNA3 | | | | | | | | |
| naked | + | + | − | − | − | − | − | − |
| siRNA | | | | | | | | |
| RBD | + | + | − | − | − | − | − | − |
| comtrol | | | | | | | | |
| positive | + | + | + | − | − | − | − | − |
| control | | | | | | | | |
| negtive | − | − | − | − | − | − | − | − |
| control | | | | | | | | |

TABLE 7a
Test results (+/−) of RNA RT-PCR viruses after co-culturing
the RBD₂-shRNA(4-8) with B.1.617.1 strain for 1 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | − | − | − | − | − | − |
| shRNA4/lip | | | | | | | | |
| RBD₂- | + | + | − | − | − | − | − | − |
| shRNA5/lip | | | | | | | | |
| RBD₂- | + | + | − | − | − | − | − | − |
| shRNA6/lip | | | | | | | | |
| RBD₂- | + | + | − | − | − | − | − | − |
| shRNA7/lip | | | | | | | | |
| RBD₂- | + | + | − | − | − | − | − | − |
| shRNA8/lip | | | | | | | | |
| naked | + | + | − | − | − | − | − | − |
| shRNA5 | | | | | | | | |
| positive | + | + | + | − | − | − | − | − |
| control | | | | | | | | |
| negtive | − | − | − | − | − | − | − | − |
| control | | | | | | | | |

TABLE 8
Test results (+/−) of RNA RT-PCR viruses after co-culturing
the compounds with B.1.617.1 strain for 24 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | − | − | − | − | − |
| shRNA1/lip | | | | | | | | |
| RBD₂- | + | + | + | + | − | − | − | − |
| shRNA2/lip | | | | | | | | |
| RBD₂- | + | + | + | − | − | − | − | − |
| shRNA3/lip | | | | | | | | |
| RBD- | + | + | + | + | − | − | − | − |
| siRNA/lip | | | | | | | | |
| naked | + | + | + | + | + | + | − | − |
| shRNA1 | | | | | | | | |
| naked | + | + | + | + | + | + | − | − |
| shRNA2 | | | | | | | | |
| naked | + | + | + | + | + | + | − | − |
| shRNA3 | | | | | | | | |
| naked | + | + | + | + | + | + | + | − |
| siRNA | | | | | | | | |
| RBD | + | + | + | + | + | − | − | − |
| comtrol | | | | | | | | |
| positive | + | + | + | + | + | + | + | − |
| control | | | | | | | | |
| negtive | − | − | − | − | − | − | − | − |
| control | | | | | | | | |

TABLE 8a
Test results (+/−) of RNA RT-PCR viruses after co-culturing
the RBD₂-shRNA(4-8) with B.1.617.1 strain for 24 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | − | − | − | − | − |
| shRNA4/lip | | | | | | | | |
| RBD₂- | + | + | + | − | − | − | − | − |
| shRNA5/lip | | | | | | | | |
| RBD₂- | + | + | + | + | − | − | − | − |
| shRNA6/lip | | | | | | | | |
| RBD₂- | + | + | + | − | − | − | − | − |
| shRNA7/lip | | | | | | | | |
| RBD₂- | + | + | + | − | − | − | − | − |
| shRNA8/lip | | | | | | | | |
| naked | + | + | + | + | + | − | − | − |
| shRNA5 | | | | | | | | |
| positive | + | + | + | + | + | + | − | − |
| control | | | | | | | | |
| negtive | − | − | − | − | − | − | − | − |
| control | | | | | | | | |

TABLE 9
Test results (+/−) of RNA RT-PCR viruses after co-culturing
the compounds with B.1.617.1 strain for 72 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | + | − | − | − | − |
| shRNA1/lip | | | | | | | | |
| RBD₂- | + | + | + | + | + | − | − | − |
| shRNA2/lip | | | | | | | | |
| RBD₂- | + | + | + | + | − | − | − | − |
| shRNA3/lip | | | | | | | | |
| RBD- | + | + | + | + | + | − | − | − |
| siRNA/lip | | | | | | | | |
| naked | + | + | + | + | + | + | + | + |
| shRNA1 | | | | | | | | |
| naked | + | + | + | + | + | + | + | − |
| shRNA2 | | | | | | | | |
| naked | + | + | + | + | + | + | + | + |
| shRNA3 | | | | | | | | |
| naked | + | + | + | + | + | + | + | + |
| siRNA | | | | | | | | |
| RBD | + | + | + | + | + | + | − | − |
| comtrol | | | | | | | | |
| positive | + | + | + | + | + | + | + | + |
| control | | | | | | | | |
| negtive | − | − | − | − | − | − | − | − |
| control | | | | | | | | |

TABLE 9a
Test results (+/−) of RBD2-shRNA (4-8) and B.1.6 viruses
RNA RT-PCR results of co-culture for 72 hours

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | + | − | − | − | − |
| shRNA4/lip | | | | | | | | |
| RBD₂- | + | + | + | + | − | − | − | − |
| shRNA5/lip | | | | | | | | |
| RBD₂- | + | + | + | + | + | − | − | − |
| shRNA6/lip | | | | | | | | |
| RBD₂- | + | + | + | + | − | − | − | − |
| shRNA7/lip | | | | | | | | |
| RBD₂- | + | + | + | + | − | − | − | − |
| shRNA8/lip | | | | | | | | |
| naked | + | + | + | + | + | + | + | − |
| shRNA5 | | | | | | | | |
| positive | + | + | + | + | + | + | + | + |
| control | | | | | | | | |
| negtive | − | − | − | − | − | − | − | − |
| control | | | | | | | | |

\textcircled{2} Test results of B.1.617.2 strains: as shown in Table 10, after the cells of each group were cultured for 1 h, the RNA of the negative control group was negative, the titer of RNA of the positive control group was 1:36, and the titer of RNA test results of all other groups was 1:12 to 1:36. As shown in Table 11, after co-culture 24 h of group, the test results of viral RNA in the negative control group was still negative, and the RNA test titer of the positive control group was 1:2916, and the RNA test results of the 4 control groups were 1:2916, while the RNA test titer of the 4 experimental groups was 1:108 to 1:324, which was significantly lower than that of the control groups (p<0.01). As shown in Table 12, after 72 h of co-culture, the negative control viral RNA remained negative, the positive control RNA titer>1:8748, the RNA test results of 4 control groups (naked) were 1:8748 or more, and in 4 experimental groups one group was 1:972 and the others were 1:324, there is still a significant difference compared with the control group (p<0.01).

The test results of RBD₂-shRNA (4-8) in Tables 10a, Table 11a and Table 12a are consistent with the test results of RBD2-shRNA (1-3) in Table 10, Table 11 and Table 122. When compared with the positive control group, all have significant antiviral effects.

Table 10˜12 shows that the experimental group had obvious effect of anti-B.1.617.2strain, indicating that shRNA or siRNA connected to the RBD could be delivered to the target cells for RNA interference, while shRNA or siRNA not connected to the RBD could not enter the target cells, thus failing to exert the role of RNA interference.

Tables 7˜12 show that the experimental group has effects of anti-B.1.617.1 and anti-B.1.617.2, shows that the compound (shRNA) targeted by the conserved gene has the effect of broad-spectrum mutation resistance strain.

TABLE 10
Test results (+/−) of RNA RT-PCR viruses after co-culturing
the compounds with B.1.617.2 strain for 1 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | − | − | − | − | − | − |
| shRNA1/lip | | | | | | | | |
| RBD₂- | + | + | − | − | − | − | − | − |
| shRNA2/lip | | | | | | | | |
| RBD₂- | + | + | + | − | − | − | − | − |
| shRNA3/lip | | | | | | | | |
| RBD- | + | + | − | − | − | − | − | − |
| siRNA/lip | | | | | | | | |
| naked | + | + | − | − | − | − | − | − |
| shRNA1 | | | | | | | | |
| naked | + | + | − | − | − | − | − | − |
| shRNA2 | | | | | | | | |
| naked | + | + | − | − | − | − | − | − |
| shRNA3 | | | | | | | | |
| naked | + | + | − | − | − | − | − | − |
| siRNA | | | | | | | | |
| RBD | + | + | − | − | − | − | − | − |
| comtrol | | | | | | | | |
| positive | + | + | + | − | − | − | − | − |
| control | | | | | | | | |
| negtive | − | − | − | − | − | − | − | − |
| control | | | | | | | | |

TABLE 10a
Test results (+/−) of RNA RT-PCR viruses after co-culturing
the RBD₂-shRNA(4-8) with B.1.617.2 strain for 1 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | − | − | − | − | − | − |
| shRNA4/lip | | | | | | | | |
| RBD₂- | + | + | − | − | − | − | − | − |
| shRNA5/lip | | | | | | | | |
| RBD₂- | + | + | + | − | − | − | − | − |
| shRNA6/lip | | | | | | | | |
| RBD₂- | + | + | − | − | − | − | − | − |
| shRNA7/lip | | | | | | | | |
| RBD₂- | + | + | − | − | − | − | − | − |
| shRNA8/lip | | | | | | | | |
| naked | + | + | − | − | − | − | − | − |
| shRNA5 | | | | | | | | |
| positive | + | + | + | − | − | − | − | − |
| control | | | | | | | | |
| negtive | − | − | − | − | − | − | − | − |
| control | | | | | | | | |

TABLE 11
Test results (+/−) of RNA RT-PCR viruses after co-culturing
the compounds with B.1.617.2 strain for 24 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | − | − | − | − | − |
| shRNA1/lip | | | | | | | | |
| RBD₂- | + | + | + | + | − | − | − | − |
| shRNA2/lip | | | | | | | | |
| RBD₂- | + | + | + | + | − | − | − | − |
| shRNA3/lip | | | | | | | | |
| RBD- | + | + | + | + | + | − | − | − |
| siRNA/lip | | | | | | | | |
| naked | + | + | + | + | + | + | + | − |
| shRNA1 | | | | | | | | |
| naked | + | + | + | + | + | + | + | − |
| shRNA2 | | | | | | | | |
| naked | + | + | + | + | + | + | − | − |
| shRNA3 | | | | | | | | |
| naked | + | + | + | + | + | + | + | − |
| siRNA | | | | | | | | |
| RBD | + | + | + | + | + | − | − | − |
| comtrol | | | | | | | | |
| positive | + | + | + | + | + | + | + | − |
| control | | | | | | | | |
| negtive | − | − | − | − | − | − | − | − |
| control | | | | | | | | |

TABLE 11a
Test results (+/−) of RNA RT-PCR viruses after co-culturing
the RBD₂-shRNA(4-8) with B.1.617.2 strain for 24 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | − | − | − | − | − |
| shRNA4/lip | | | | | | | | |
| RBD₂- | + | + | + | − | − | − | − | − |
| shRNA5/lip | | | | | | | | |
| RBD₂- | + | + | + | + | − | − | − | − |
| shRNA6/lip | | | | | | | | |
| RBD₂- | + | + | + | − | − | − | − | − |
| shRNA7/lip | | | | | | | | |
| RBD₂- | + | + | + | − | − | − | − | − |
| shRNA8/lip | | | | | | | | |
| naked | + | + | + | + | + | + | − | − |
| shRNA5 | | | | | | | | |
| positive | + | + | + | + | + | + | + | + |
| control | | | | | | | | |
| negtive | − | − | − | − | − | − | − | − |
| control | | | | | | | | |

TABLE 12
Test results (+/−) of RNA RT-PCR viruses after co-culturing
the compounds with B.1.617.2 strain for 72 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | + | + | − | − | − |
| shRNA1/lip | | | | | | | | |
| RBD₂- | + | + | + | + | + | − | − | − |
| shRNA2/lip | | | | | | | | |
| RBD₂- | + | + | + | + | + | + | − | − |
| shRNA3/lip | | | | | | | | |
| RBD- | + | + | + | + | + | − | − | − |
| siRNA/lip | | | | | | | | |
| naked | + | + | + | + | + | + | + | + |
| shRNA1 | | | | | | | | |
| naked | + | + | + | + | + | + | + | + |
| shRNA2 | | | | | | | | |
| naked | + | + | + | + | + | + | + | − |
| shRNA3 | | | | | | | | |
| naked | + | + | + | + | + | + | + | + |
| siRNA | | | | | | | | |
| RBD | + | + | + | + | + | − | − | − |
| comtrol | | | | | | | | |
| positive | + | + | + | + | + | + | + | − |
| control | | | | | | | | |
| negtive | − | − | − | − | − | − | − | − |
| control | | | | | | | | |

TABLE 12a
Test results (+/−) of RNA RT-PCR viruses after co-culturing
the RBD₂-shRNA(4-8) with B.1.617.2 strain for 72 hour.

| | test results (+/−) of RNA viruses at | | | | | | | |
|---|---|---|---|---|---|---|---|---|
| | different dilution times of culture medium | | | | | | | |
| group | 1:4 | 1:12 | 1:36 | 1:108 | 1:324 | 1:972 | 1:2916 | 1:8748 |
| RBD₂- | + | + | + | + | + | − | − | − |
| shRNA4/lip | | | | | | | | |
| RBD₂- | + | + | + | + | − | − | − | − |
| shRNA5/lip | | | | | | | | |
| RBD₂- | + | + | + | + | + | − | − | − |
| shRNA6/lip | | | | | | | | |
| RBD₂- | + | + | + | + | − | − | − | − |
| shRNA7/lip | | | | | | | | |
| RBD₂- | + | + | + | + | − | − | − | − |
| shRNA8/lip | | | | | | | | |
| naked | + | + | + | + | + | + | + | − |
| shRNA5 | | | | | | | | |
| positive | + | + | + | + | + | + | + | + |
| control | | | | | | | | |
| negtive | − | − | − | − | − | − | − | − |
| control | | | | | | | | |

### 2. Verification of Targeted Delivery and Immune Function of RBD in Animals

### (1) Group Grouping and Inoculation of Animals

Group of animals: SPF female BALB/c mice aged from 6 weeks to 8 weeks and about 40 grams were selected and randomly divided them into RBD2-shRNA1 (RBD2-shRNA 1-8)/Lip group (inoculation with RBD2-shRNA1/Lip+B.1.617.2 strains), RBD-siRNA1/Lip group (vaccinated with RBD-siRNA1/Lip+B.1.617.2 strains), RBD group (vaccinated with RBD+B.1.617.2 strains), shRNA1/Lip group (inoculation with shRNA1/Lip+B.1.617.2 strains), group shRNA1 (inoculated with shRNA1+B.1.617.2 strains) positive control group (inoculation B.1.617.2 plant+saline) and the negative control group (inoculated with saline only).

Animal inoculation: nasal spray inoculation a 40 μl the B.1.617.2 viruses solution with a titer of 105/mlTCID₅₀, the negative control group was vaccinated with 40 μl saline by nasal spray. They were anesthetized with intraperitoneal injection of 5% chloral solution; and 0.1 nmol of RBD2-shRNA1/Lip, RBD-siRNA1/Lip, RBD, shRNA1/Lip and shRNA1 were slowly injected into the mouse trachea, tissue reset, 10 mice in each group were killed at 7^{th} day after infection for viruses detection, and another 10 mice were used to visualize antibodies.

### (2) Detecting Viruses by a Percent of Median Tissue Culture Infectious Dose (TCID₅₀)

Preparing 10% homogenate from lung tissue of euthanized mice, centrifuged at 100 pl; taking 100 pl of centrifuged supernatant fluid; and diluting in 10 times increments; and inoculated on a 96-well plates grown of VeroE6 monolayer growth, 30 μl each well, 4 wells for per dilution degree; and gently shaking and homogenized, and adsorbing at 37°° C. for 1 h; and washing with Hank's solution; and adding culture medium; and incubating in an incubator at 37° C. CO₂; Observing the cytopathic effect (CPE); and calculating the median tissue culture infectious dose (TCID50). Where the higher the percentage, the higher the virus content, shown in table 13˜19.

TABLE 13
Percentage of VeroE6 median tissue culture infectious
dose of lung tissue homogenate of euthanized mice
of the RBD₂-shRNA1/Lip group

| Dilution | Inoculated | | | | |
|---|---|---|---|---|---|
| degree of | cell pore | Total Observations | | Infec- | Infec- |
| tissue | count(4cell × | Normal | Infected | tion | tion |
| homogenate | 10cases) | cell | cell | ratio | percent |
| 10¹ | 40 | 32 | 8 | 8/40 | 20.0 |
| 10² | 40 | 35 | 5 | 5/40 | 12.5 |
| 10³ | 40 | 37 | 3 | 3/40 | 7.5 |
| 10⁴ | 40 | 38 | 2 | 2/40 | 5.0 |

TABLE 14
Percentage of VeroE6 median tissue culture infectious
dose of lung tissue homogenate of euthanized
mice of the RBD-siRNA1/Lip group

| Dilution | Inoculated | | | | |
|---|---|---|---|---|---|
| degree of | cell pore | Total Observations | | Infec- | Infec- |
| tissue | count(4cell × | Normal | Infected | tion | tion |
| homogenate | 10cases) | cell | cell | ratio | percent |
| 10¹ | 40 | 29 | 11 | 11/40 | 27.5 |
| 10² | 40 | 32 | 8 | 8/40 | 20.0 |
| 10³ | 40 | 34 | 6 | 6/40 | 15.0 |
| 10⁴ | 40 | 36 | 4 | 4/40 | 10.0 |

TABLE 15
Percentage of VeroE6 median tissue culture infectious dose of
lung tissue homogenate of euthanized mice of the RBD group

| Dilution | Inoculated | | | | |
|---|---|---|---|---|---|
| degree of | cell pore | Total Observations | | Infec- | Infec- |
| tissue | count(4cell × | Normal | Infected | tion | tion |
| homogenate | 10cases) | cell | cell | ratio | percent |
| 10¹ | 40 | 5 | 35 | 35/40 | 87.5 |
| 10² | 40 | 8 | 32 | 32/40 | 80.0 |
| 10³ | 40 | 13 | 27 | 27/40 | 67.5 |
| 10⁴ | 40 | 19 | 21 | 21/40 | 52.5 |

TABLE 16
Percentage of VeroE6 median tissue culture infectious dose of lung
tissue homogenate of euthanized mice of the shRNA1/Lip group

| Dilution | Inoculated | | | | |
|---|---|---|---|---|---|
| degree of | cell pore | Total Observations | | Infec- | Infec- |
| tissue | count(4cell × | Normal | Infected | tion | tion |
| homogenate | 10cases) | cell | cell | ratio | percent |
| 10¹ | 40 | 7 | 33 | 33/40 | 82.5 |
| 10² | 40 | 9 | 31 | 31/40 | 77.5 |
| 10³ | 40 | 15 | 25 | 25/40 | 62.5 |
| 10⁴ | 40 | 21 | 19 | 19/40 | 47.5 |

TABLE 17
Percentage of VeroE6 median tissue culture infectious dose of
lung tissue homogenate of euthanized mice of the shRNA1 group

| Dilution | Inoculated | | | | |
|---|---|---|---|---|---|
| degree of | cell pore | Total Observations | | Infec- | Infec- |
| tissue | count(4cell × | Normal | Infected | tion | tion |
| homogenate | 10cases) | cell | cell | ratio | percent |
| 10¹ | 40 | 2 | 38 | 38/40 | 95.0 |
| 10² | 40 | 3 | 37 | 36/40 | 90.0 |
| 10³ | 40 | 7 | 33 | 33/40 | 82.5 |
| 10⁴ | 40 | 12 | 28 | 28/40 | 70.0 |

TABLE 18
Percentage of VeroE6 median tissue culture infectious dose of lung
tissue homogenate of euthanized mice of the positive control mice

| Dilution | Inoculated | | | | |
|---|---|---|---|---|---|
| degree of | cell pore | Total Observations | | Infec- | Infec- |
| tissue | count(4cell × | Normal | Infected | tion | tion |
| homogenate | 10cases) | cell | cell | ratio | percent |
| 10¹ | 40 | 2 | 38 | 38/40 | 95.0 |
| 10² | 40 | 3 | 37 | 37/40 | 92.5 |
| 10³ | 40 | 4 | 36 | 36/40 | 90.0 |
| 10⁴ | 40 | 6 | 34 | 34/40 | 85.0 |

TABLE 19
Percentage of VeroE6 median tissue culture infectious dose of lung
tissue homogenate of euthanized mice of the negative control mice

| Dilution | Inoculated | | | | |
|---|---|---|---|---|---|
| degree of | cell pore | Total Observations | | Infec- | Infec- |
| tissue | count(4cell × | Normal | Infected | tion | tion |
| homogenate | 10cases) | cell | cell | ratio | percent |
| 10¹ | 40 | 39 | 1 | 1/40 | 2.5 |
| 10² | 40 | 39 | 1 | 1/40 | 2.5 |
| 10³ | 40 | 38 | 2 | 2/40 | 5.0 |
| 10⁴ | 40 | 37 | 3 | 3/40 | 7.5 |

### (3) The Effects of the RBD Targeted Delivery

Refers Tables 13 to 19, Percentage of VeroE6 median tissue culture infectious dose of lung tissue homogenate of euthanized mice is 20.0% in RBD₂-shRNA1/Lip group, 27.5% in RBD-siRNA1/Lip group, 87.5% in RBD group, 82.5% in shRNA1/Lip group, 95.0% in shRNA1 group, 95.0% in positive control group and 2.5% in negative control group. Because RNAi mainly occurs in the cytoplasm, the shRNA1 in the shRNA1 group is easily degraded by nuclease and difficult to cross the cell membrane, so it hardly plays an RNAi role. Although the shRNA1 in the shRNA1/Lip group is protected by Lip and hardly degraded by nuclease and can pass through the cell membrane, the RNAi is inability to specifically enter target cells, resulting in poor RNAi efficacy, and the percentage of VeroE6 infection is 82.5%, no significant difference with the positive control group (p>0.05); and the shRNA1/siRNA1 in the RBD₂-shRNA1/Lip group and the RBD-siRNA1/Lip group were targeted by RBD, and RNAi efficacy is good; the percentage of VeroE6 TCID₅₀ in the the RBD2-shRNA1/Lip group and the RBD-siRNA1/Lip group was significantly different compared with the shRNA1/Lip group (p<0.05).

Referring to the TCID₅₀ test method mentioned above, the shRNA7 and shRNA8 had high silencing efficiency (90% and 91%, respectively), and RBD₂-shRNA7/lip group and the RBD₂-shRNA8/lip group was selected to perform TCID₅₀ test. And the results of the TCID₅₀ of the RBD₂-shRNA7/lip group and the RBD₂-shRNA8/lip, positive control group and negative control group are 22.5%, 22.5%, 92.5%, and 5.0%, respectively, the TCID₅₀ test results of the experimental groups were significantly lower than those found in the positive control group.

(4) Immune Function Detection of the Compound (nCOVsiRNA)

Collecting RBD2For venous blood from 10 mice at 1 week, 2 weeks, 4 weeks, and 6 weeks after inoculation in the-shRNA1/Lip and RBD groups, serum was isolated and stored at minus 20° C., operated by kit and tested for specific antibodies IgG and IgM by ELISA (Table 20).

TABLE 20
Comparison of serum specific antibody detection results
in 10 mice of the RBD₂-shRNA1/Lip group and RBD group.

| | Antibody positive serum (case/time) | | | | | |
|---|---|---|---|---|---|---|
| | IgM | | IgG | | IgM + IgG | |
| | RBD₂- | | RBD₂- | | RBD₂- | |
| | shRNA1/ | | shRNA1/ | | shRNA1/ | |
| Time(week) | Lip | RBD | Lip | RBD | Lip | RBD |
| 1 | 4 | 2 | 2 | 0 | 2 | 0 |
| 2 | 7 | 3 | 5 | 2 | 5 | 2 |
| 4 | 6 | 2 | 7 | 3 | 6 | 2 |
| 6 | 4 | 1 | 6 | 3 | 3 | 1 |
| Total | 21* | 8 | 20* | 8 | 16* | 5 |

As shown in Table 20, the detected cases of IgM, IgG and IgM+IgG in the RBD₂-shRNA1/Lip group were 21 cases, 20 cases, and 16 cases, respectively, more than 8 cases, 8 cases and 5 cases in the RBD group, respectively. This is because RBD2-shRNA1/Li group is synthesized by 2 parts of RBD, 1 part of shRNA and Lip, which have larger molecular weight and more complex molecular structure than the single RBD in the RBD group. Further, shRNA and Lip have the effect of immune adjuvant, so they are more antigenic and more likely to produce antibodies.

Although the above embodiments give a detailed description of the disclosure, it is only one rather not all embodiments, and one may obtain others without creativity, all of which fall within the scope of protection of the disclosure.

1. A nCOVsiRNA drug for targeted delivery of shRNA by RBD, wherein the nCOVsiRNA drug is synthesized by the following steps:
synthesizing shRNA and RBD polypeptide of coronavirus, wherein the shRNA is common targeted siRNA of Covid-19 strains; and ACE2 is adopted as receptors of the RBD;
connecting the RBD polypeptide to plus and antisense strands of the shRNA, respectively, to obtain a RBD-shRNA-RBD compound for targeted delivery of the shRNA to ACE2 by RBD; and
modifing the RBD-shRNA-RBD compound with liposome, to obtain a nCoVshRNA⋅2RBD drug.

2. The nCOVsiRNA drug for targeted delivery of shRNA by RBD according to claim 1, wherein, the shRNA is played a specific role against ACE2 expressing cells, and a mutual efficiency of components is increased by the nCoVshRNA⋅2RBD drug, which is caused by the targeted delivery of RBD; and
the RBD polypeptide is protein antigen, and RBD-dimer vaccine is prepared by connecting one part of the shRNA with two part of the RBD, and molecular structure and molecular weight are changed, and an immune effect of RBD-dimer vaccine is enhanced;
the RBD polypeptide is a kind of cell penetrating peptide, a cellular membrane permeability and a antinuclease stability are enhanced by connecting the shRNA to the RBD;
the RBD polypeptide is combined with the ACE2 receptors and limited viral RBD to bind to the ACE2 receptors, to inhibit viruses infection;
the shRNA and the liposome are immune adjuvants to enhance the immune effect of RBD-dimer vaccine.

3. The nCOVsiRNA drug for targeted delivery of shRNA by RBD according to claim 1, wherein, the common targeted siRNA of Covid-19 strains is common conserved genes which are selected from various pathogenic coronavirus and related variant strains that recorded on database;
interfering the common conserved genes by the shRNA, to act as a broad spectrum of anti variant strains;
the common conserved genes comprise but are not limited to ultraconserved genes, conserved genes, and/or genes spliced by conserved microsatellite gene.

4. The nCOVsiRNA drug for targeted delivery of shRNA by RBD according to claim 1, wherein, the step of synthesizing shRNA comprises the following step:
Synthesizing two complementary oligonucleotides based on selected siRNA, where the nucleotide number of each complementary oligonucleotide is 21 nt to 25 nt;
synthesizing base sequences to act as spacers; and
synthesizing small hairpin shRNA duplexes by the two complementary oligonucleotides and the base sequences, where, a loop ring of the small hairpin shRNA duplex is formed by the base sequences.

5. The nCOVsiRNA drug for targeted delivery of shRNA by RBD according to claim 3, wherein a sequence of the common target siRNA comprises is one sequence selected from SEQ ID NO. 1 to 58; or
sequences of the common target siRNA comprises two or more sequences selected from SEQ ID NO. 1 to 58.

6. The nCOVsiRNA drug for targeted delivery of shRNA by RBD according to claim 3, wherein a sequence of the common target siRNA comprises one sequence selected from SEQ ID NO. 1, SEQ ID NO. 2, SEQ ID NO. 5, SEQ ID NO. 7 to 10, SEQ ID NO. 16 to 18, SEQ ID NO. 20 to 22, SEQ ID NO. 30 to 32, SEQ ID NO. 41 to 58; or
sequences of the common target siRNA comprise two or more sequences selected from SEQ ID NO. 1, SEQ ID NO. 2, SEQ ID NO. 5, SEQ ID NO. 7 to 10, SEQ ID NO. 16 to 18, SEQ ID NO. 20 to 22, SEQ ID NO. 30 to 32, SEQ ID NO. 41 to 58.

7. The nCOVsiRNA drug for targeted delivery of shRNA by RBD according to claim 3 wherein a sequence of the common target siRNA comprises one sequence selected from SEQ ID NO. 1, SEQ ID NO. 2, SEQ ID NO. 5, SEQ ID NO. 8, SEQ ID NO. 17, SEQ ID NO. 30, SEQ ID NO. 41 and SEQ ID NO. 50; or
sequences of the common target siRNA comprise two or more sequences selected from SEQ ID NO. 1, SEQ ID NO. 2, SEQ ID NO. 5, SEQ ID NO. 8, SEQ ID NO. 17, SEQ ID NO. 30, SEQ ID NO. 41 and SEQ ID NO. 50.

8. The nCOVsiRNA drug for targeted delivery of shRNA by RBD according to claim 1, wherein, the RBD polypeptide is an amino acid sequence binding with ACE2;
wherein the RBD polypeptide comprises 319th to 401th amino acid sequence of S protein, conserved amino acid sequence at sites of N439, V483 and Q493, and codon-optimized amino acid sequence.

9. The nCOVsiRNA drug for targeted delivery of shRNA by RBD according to claim 1, wherein, connecting ways in the step of connecting the RBD polypeptide to plus and antisense strands of the shRNA comprise:
connecting glycosylated N-amino of the RBD polypeptide with terminal of 3′ antisense strand, terminal of 5′ or 3′ of plus strand; and
chemical coupling or covalent coupling by disulfide bond, phosphodiester bond, dithiophosphorate lipid bond, sulfide bond, oxime bond, amide bond or maleimide-thiol bond.

10. The nCOVsiRNA drug for targeted delivery of shRNA by RBD according to claim 1, wherein the nCOVsiRNA drug comprises a binder RBD-siRNA and S-siRNA connected to the RBD polypeptide or S protein polypeptide, and one or several of liposomal modification compound of RBD-siRNA/Lip and S-siRNA/Lip.

11. A synthesis method of a chemical drug nCoVshRNA⋅2RBD, wherein, comprising following steps of:
synthesizing shRNA by plus and antisense strands of siRNA with RNAi function;
connecting targeted delivery carrier RBD to terminals of siRNA plus strand and siRNA antisense strand of the shRNA, to obtain a nCoVshRNA⋅2RBD drug.

12. A synthesis method of a chemical drug nCoVshRNA⋅2RBD according to claim 11, wherein, the siRNA is selected from common sequences of RNAi of various pathogenic coronavirus and related variant strains;
wherein the shRNA is configured to interface viruses that contain the common sequences of RNAi anti variant strains, and to generate broad spectrum RNAi effects against mutant strains.

13. A synthesis method of a chemical drug nCoVshRNA⋅2RBD according to claim 11, wherein, the step of synthesizing shRNA by plus and antisense strands of siRNA comprises following steps:
synthesizing two complementary oligonucleotides based on selected siRNA, where the nucleotide number of each complementary oligonucleotide is 21 nt to 25 nt
synthesizing base sequences to act as spacers; and
synthesizing small hairpin shRNA duplexes by the two complementary oligonucleotides and the base sequences, where, a loop ring of the small hairpin shRNA duplex is formed by the base sequences.

14. A synthesis method of a chemical drug nCoVshRNA⋅2RBD according to claim 11, wherein, wherein a sequence of the common target siRNA comprises is one sequence selected from SEQ ID NO. 1 to 58; or
sequences of the common target siRNA comprises two or more sequences selected from SEQ ID NO. 1 to 58.

15. A synthesis method of a chemical drug nCoVshRNA⋅2RBD according to claim 11, wherein a sequence of the common target siRNA comprises one sequence selected from SEQ ID NO. 1, SEQ ID NO. 2, SEQ ID NO. 5, SEQ ID NO. 7 to 10, SEQ ID NO. 16 to 18, SEQ ID NO. 20 to 22, SEQ ID NO. 30 to 32, SEQ ID NO. 4 to 58; or
sequences of the common target siRNA comprise two or more sequences selected from SEQ ID NO. 1, SEQ ID NO. 2, SEQ ID NO. 5, SEQ ID NO. 7 to 10, SEQ ID NO. 16 to 18, SEQ ID NO. 20 to 22, SEQ ID NO. 30 to 32, SEQ ID NO. 41 to 58.

16. A synthesis method of a chemical drug nCoVshRNA⋅2RBD according to claim 11, wherein a sequence of the common target siRNA comprises one sequence selected from SEQ ID NO. 16 to 18 and SEQ ID NO. 49 to 51 of targeting coronavirus N gene, SEQ ID NO. 20 to 22 and SEQ ID NO. 52 to 54 of targeting coronavirus OFR1ab gene, and SEQ ID NO. 30 to 32 and SEQ ID NO. 56 to 58 of targeting coronavirus S gene; or
sequences selected from SEQ ID NO. 16 to 18 and SEQ ID NO. 49 to 51 of targeting coronavirus N gene, SEQ ID NO. 20 to 22 and SEQ ID NO. 52 to 54 of targeting coronavirus OFR1ab gene, and SEQ ID NO. 30 to 32 and SEQ ID NO. 56 to 58 of targeting coronavirus S gene.

17. A synthesis method of a chemical drug nCoVshRNA⋅2RBD according to claim 11, wherein the siRNA adopted to synthesize shRNA comprises one sequence selected from SEQ ID NO. 16 and SEQ ID NO. 49 of targeting coronavirus N gene, SEQ ID NO. 21 and SEQ ID NO. 52 of targeting coronavirus OFR1ab gene, and SEQ ID NO. 30 of targeting coronavirus S gene.

18. A synthesis method of a chemical drug nCoVshRNA⋅2RBD according to claim 11, wherein the RBD polypeptide is an amino acid sequence binding with ACE2;
wherein the RBD polypeptide comprises 319th to 401th amino acid sequence of S protein, conserved amino acid sequence at sites of N439, V483 and Q493, and codon-optimized amino acid sequence.

19. A synthesis method of a chemical drug nCoVshRNA⋅2RBD according to claim 11, wherein connecting ways in the step of connecting the RBD polypeptide to plus and antisense strands of the shRNA comprise:
connecting glycosylated N-amino of the RBD polypeptide with terminal of 3′ antisense strand, terminal of 5′ or 3′ of plus strand; and
chemical coupling or covalent coupling by disulfide bond, phosphodiester bond, dithiophosphorate lipid bond, sulfide bond, oxime bond, amide bond or maleimide-thiol bond.

20. The application of a chemical drug nCoVshRNA⋅2RBD, wherein, an application of a chemical drug nCoVshRNA⋅2RBD is to prepare RBD vaccine;
wherein the shRNA has an immune adjuvant effect, and the RBD polypeptide have effects of broading spectrum anti-mutation strain and protein antigen.
